SEQUENCE LISTING (25 sequences)

SEQ ID NO: 1 - protein, 362 aa, synthetic construct
  REGION 1..362: note = CSQ1
QEGLDFPEYDGVDRVINVNAKNYKNVFKKYEVLALLYHEPPEDDKASQRQFEMEELILELAAQVLEDKGVGFGLVDSEKDAAVAKKLGLTEVDSMYVFKGDEVIEYDGEFSADTIVEFLLDVLEDPVELIEGERELQAFENIEDEIKLIGYFKSKDSEHYKAFEDAAEEFHPYIPFFATFDSKVAKKLTLKLNEIDFYEAFMEEPVTIPDKPNSEEEIVNFVEEHRRSTLRKLKPESMYETWEDDMDGIHIVAFAEEADPDGFEFLETLKAVAQDNTENPDLSIIWIDPDDFPLLVPYWEKTFDIDLSAPQIGVVNVTDADSVWMEMDDEEDLPSAEELEDWLEDVLEGEINTEDDDDDDDD

SEQ ID NO: 2 - protein, 380 aa, synthetic construct
  REGION 1..380: note = CSQ2
EEGLNFPTYDGKDRVVSLSEKNFKQVLKKYDLLCLYYHEPVSSDKVTPKQFQLKEIVLELVAQVLEHKAIGFVMVDAKKEAKLAKKLGFDEEGSLYILKGDRTIEFDGEFAADVLVEFLLDLIEDPVEIISSKLEVQAFERIEDYIKLIGFFKSEDSEYYKAFEEAAEHFQPYIKFFATFDKGVAKKLSLKMNEVDFYEPFMDEPIAIPNKPYTEEELVEFVKEHQRPTLRRLRPEEMFETWEDDLNGIHIVAFAEKSDPDGYEFLEILKQVARDNTDNPDLSILWIDPDDFPLLVAYWEKTFKIDLFRPQIGVVNVTDADSVWMEIPDDDDLPTAEELEDWIEDVLSGKINTEDDDEDDDDDDNSDEEDNDDSDDDDDE

SEQ ID NO: 3 - DNA, 1086 bp, synthetic construct
  misc_feature 1..1086: note = CSQ1
  source 1..1086: mol_type = other DNA
caggaagggctggacttccctgagtacgatggtgtggaccgtgtgatcaatgtcaatgcaaagaactacaagaatgtgttcaagaagtatgaggtgctggcactcctctaccatgaaccccccgaggatgacaaggcctcacaaagacaatttgagatggaggagctgatcctggagttagcagcccaagtcctagaagacaagggtgttggcttcgggctggtagactctgagaaggatgcagctgtggccaagaaactaggcctaactgaagtggacagcatgtatgtattcaagggagatgaagtcattgagtacgatggcgagttttctgctgacaccatcgtggagtttctgcttgatgtcctagaggaccctgtggaattgattgaaggtgaacgagagctgcaggcgtttgagaatattgaggatgagatcaaactcattggctacttcaagagcaaagactcagagcattacaaagccttcgaggatgcagctgaggagtttcatccctacatccccttcttcgccaccttcgacagcaaggtggcaaagaagctgaccctgaagctgaatgagattgatttctacgaggccttcatggaagagcctgtgaccatcccagacaagcccaatagcgaagaggagattgtcaacttcgtggaggagcacaggagatcaaccctgaggaaactgaagccggagagtatgtatgagacctgggaggatgatatggatggaatccacattgtggccttcgcagaggaagctgatcctgatggtttcgagttcttagagactctcaaggctgtggcccaagataacactgaaaacccagatcttagcatcatctggattgaccctgatgacttccccctgctggtcccatactgggagaagacgtttgacatcgacttgtcagccccacaaataggagtcgtcaatgttactgatgcggatagcgtatggatggaaatggacgatgaggaggacctgccttctgctgaggagctggaggactggctggaggatgtcctggagggcgagatcaacacagaggacgatgacgatgatgatgatgac

SEQ ID NO: 4 - DNA, 1143 bp, synthetic construct
  misc_feature 1..1143: note = CSQ2
  source 1..1143: mol_type = other DNA
gaagaggggcttaatttccccacatatgatgggaaggaccgagtggtaagtctttccgagaagaacttcaagcaggttttaaagaaatatgacttgctttgcctctactaccatgagccggtgtcttcagataaggtcacgccaaaacagttccaactgaaagaaatcgtgcttgagcttgtggcccaggtccttgaacataaagctataggctttgtgatggtggatgccaagaaagaagccaagcttgccaagaaactgggttttgatgaagaaggaagcctgtatattcttaagggtgatcgcacaatagagtttgatggcgagtttgcagctgatgtcttggtggagttcctcttggatctaattgaagacccagtggagatcatcagcagcaaactggaagtccaagccttcgaacgcattgaagactacatcaaactcattggctttttcaagagtgaggactcagaatactacaaggcttttgaagaagcagctgaacacttccagccttacatcaaattctttgccacctttgacaaaggggttgcaaagaaattatctttgaagatgaatgaggttgacttctatgagccatttatggatgagcccattgccatccccaacaaaccttacacagaagaggagctggtggagtttgtgaaggaacaccaaagacccactctacgtcgcctgcgcccagaagaaatgtttgaaacatgggaagatgatttgaatgggatccacattgtggcctttgcagagaagagtgatccagatggctacgaattcctggagatcctgaaacaggttgcccgggacaatactgacaaccccgatctgagcatcctgtggatcgacccggacgactttcctctgctcgttgcctactgggagaagactttcaagattgacctattcaggccacagattggggtggtgaatgtcacagatgctgacagtgtctggatggagattccagatgatgacgatcttccaactgctgaggagctggaggactggattgaggatgtgctttctggaaagataaacactgaagatgatgatgaagatgatgatgatgatgataattctgatgaagaggataatgatgacagtgatgacgatgatgatgaatag

SEQ ID NO: 5 - protein, 7 aa, synthetic construct
  REGION 1..7: note = TEV
ENLYFQG

SEQ ID NO: 6 - protein, 6 aa, synthetic construct
  REGION 1..6: note = Thrombin
LVPRGS

SEQ ID NO: 7 - protein, 431 aa, synthetic construct
  REGION 1..431: note = Ssp Dna
GCISGDSLISLASTGKRVSIKDLLDEKDFEIWAINEQTMKLESAKVSRVFCTGKKLVYILKTRLGRTIKATANHRFLTIDGWKRLDELSLKEHIALPRKLESSSLQLMSDEELGLLGHLIGDGCTLPRHAIQYTSNKIELAEKVVELAKAVFGDQINPRISQERQWYQVYIPASYRLTHNKKNPITKWLENLDVFGLRSYEKFVPNQVFEQPQRAIAIFLRHLWSTDGCVKLIVEKSSRPVAYYATSSEKLAKDVQSLLLKLGINARLSKISQNGKGRDNYHVTITGQADLQIFVDQIGAVDKDKQASVEEIKTHIAQHQANTNRDVIPKQIWKTYVLPQIQIKGITTRDLQMRLGNAYCGTALYKHNLSRERAAKIATITQSPEIEKLSQSDIYWDSIVSITETGVEEVFDLTVPGPHNFVANDIIVHNS

SEQ ID NO: 8 - protein, 200 aa, synthetic construct
  REGION 1..200: note = GyrA
YCITGDALVALPEGESVRIADIVPGARPNSDNAIDLKVLDRHGNPVLADRLFHSGEHPVYTVRTVEGLRVTGTANHPLLCLVDVAGVPTLLWKLIDEIKPGDYAVIQRSAFSVDCAGFARGKPEFAPTTYTVGVPGLVRFLEAHHRDPDAQAIADELTDGRFYYAKVASVTDAGVQPVYSLRVDTADHAFITNGFVSHNT

SEQ ID NO: 9 - protein, 91 aa, synthetic construct
  REGION 1..91: note = EBD14
EVPQLTDLSFVDITDSSIGLRWVQCQCAGIIGYRITVVAAGEGIPIFEDFVDPACVYYTVTGLEPGIDYDISVITLIHRIHPNPTTLTQQT

SEQ ID NO: 10 - protein, 91 aa, synthetic construct
  REGION 1..91: note = EBD21
EVPQLTDLSFVDMTDSSIGLRWVQVMRMGIIGYRITVVAAGEGIPIFEDFVDHPIRYYTVTGLEPGIDYDISVITLIPWQRHRPTTLTQQT

SEQ ID NO: 11 - protein, 91 aa, synthetic construct
  REGION 1..91: note = EBD26
EVPQLTDLSFVDITDSSIGLRWRMTCLCLIIGYRITVVAAGEGIPIFEDFVDDPWTYYTVTGLEPGIDYDISVITLITQKHVKPTTLTQQT

SEQ ID NO: 12 - protein, 53 aa, synthetic construct
  REGION 1..53: note = EGF
NSDSECPLSHDGYCLHDGVCMYIEALDKYACNCVVGYIGERCQYRDLKWWELR

SEQ ID NO: 13 - protein, 164 aa, synthetic construct
  REGION 1..164: note = KGF1
MCNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT

SEQ ID NO: 14 - protein, 147 aa, synthetic construct
  REGION 1..147: note = VEGF
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRR

SEQ ID NO: 15 - protein, 146 aa, synthetic construct
  REGION 1..146: note = FGF2
PALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 16 - protein, 115 aa, synthetic construct
  REGION 1..115: note = BMP2
MQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 17 - protein, 113 aa, synthetic construct
  REGION 1..113: note = TGF
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCSR

SEQ ID NO: 18 - protein, 323 aa, synthetic construct
  REGION 1..323: note = HRP
QLTPTFYDNSCPNVSNIVRDTIVNELRSDPRIAASILRLHFHDCFVNGCDASILLDNTTSFRTEKDAFGNANSARGFPVIDRMKAAVESACPRTVSCADLLTIAAQQSVTLAGGPSWRVPLGRRDSLQAFLDLANANLPAPFFTLPQLKDSFRNVGLNRSSDLVALSGGHTFGKNQCRFIMDRLYNFSNTGLPDPTLNTTYLQTLRGLCPLNGNLSALVDFDLRTPTIFDNKYYVNLEEQKGLIQSDQELFSSPNATDTIPLVRSFANSTQTFFNAFVEAMDRMGNITPLTGTQGQIRLNCRVVNSNSLLHDMVEVVDFVSSM

SEQ ID NO: 19 - protein, 32 aa, synthetic construct
  REGION 1..32: note = GLP1
AAHGEGTFTSDVSSYLEEQAAKEFIAWLVKGA

SEQ ID NO: 20 - DNA, 30 bp, synthetic construct
  misc_feature 1..30: note = PRIMER
  source 1..30: mol_type = other DNA
aatggatccgaggtgccccaactcactgac

SEQ ID NO: 21 - DNA, 36 bp, synthetic construct
  misc_feature 1..36: note = PRIMER
  source 1..36: mol_type = other DNA
attctcgagttacgtttgttgtgtcagtgtagtagg

SEQ ID NO: 22 - DNA, 30 bp, synthetic construct
  misc_feature 1..30: note = PRIMER
  source 1..30: mol_type = other DNA
aatggatccaactctgatagcgaatgcccg

SEQ ID NO: 23 - DNA, 30 bp, synthetic construct
  misc_feature 1..30: note = PRIMER
  source 1..30: mol_type = other DNA
attctcgagttaacgcagttcccaccattt

SEQ ID NO: 24 - DNA, 21 bp, synthetic construct
  misc_feature 1..21: note = PRIMER
  source 1..21: mol_type = other DNA
cagcaagacagcgatggatcc

SEQ ID NO: 25 - DNA, 21 bp, synthetic construct
  misc_feature 1..21: note = PRIMER
  source 1..21: mol_type = other DNA
cgacttacaggtgatctcgag

(19) United States
(12) Patent Application Publication
(10) Pub. No.: US 2024/0209046 A1
(43) Pub. Date: Jun. 27, 2024

(54) METHOD FOR EXPRESSING AND PURIFYING PROTEIN BY USING CSQ-TAG

(71) Applicants: Korea Institute of Ceramic Engineering and Technology, Gyeongsangnam-do (KR); ToolBio. Co, Sejong-si (KR)

(72) Inventors: Sung Hyun Kim, Sejong-si (KR); Jin Ho Bang, Sejong-si (KR); Mi Hyun Choi, Busan (KR); Seho Park, Sejong-si (KR)

(73) Assignees: Korea Institute of Ceramic Engineering and Technology, Gyeongsangnam-do (KR); ToolBio. Co, Sejong-si (KR)

(21) Appl. No.: 18/453,221

(22) Filed: Aug. 21, 2023

Related U.S. Application Data
(63) Continuation-in-part of application No. 17/601,125, filed on Oct. 4, 2021.

(30) Foreign Application Priority Data
Apr. 5, 2019 (KR) 10-2019-0040241
Mar. 31, 2020 (KR) 10-2020-0039185

(51) Int. Cl. C07K 14/47; C07K 1/30; C07K 14/475; C07K 14/485; C07K 14/50; C07K 14/51; C07K 14/605; C07K 14/745; C07K 14/78; C12N 9/08; C12N 9/50; C12N 15/70; C12P 21/02

(52) U.S. Cl. CPC C07K 14/4728; C07K 1/303; C07K 14/475; C07K 14/485; C07K 14/50; C07K 14/51; C07K 14/605; C07K 14/745; C07K 14/78; C12N 9/0065; C12N 9/506; C12N 15/70; C12P 21/02; C12Y 111/01007; C12Y 304/22044; C07K 2319/20

(57) ABSTRACT

The present disclosure relates to a method for expressing, water-solubilizing, and purifying protein by using a calsequestrin-tag (CSQ-tag). Also provided are: a recombinant expression vector containing a CSQ-tag and a target protein; a host cell transformed using the recombinant expression vector; and a method for expressing and purifying a target protein by using a CSQ tag.

### CROSS-REFERENCE TO RELATED APPLICATION

The present application is a Continuation In-Part of Application of U.S. application Ser. No. 17/601,125, filed on Oct. 4, 2021, which claims priority to PCT International Application No. PCT/KR2020/004463, filed on Apr. 1, 2020, which claims priority to Korean Patent Application No. 10-2020-0039185, filed on Mar. 31, 2020 and Korean Patent Application No. 10-2019-0040241, filed on Apr. 5, 2019, which are incorporated herein by reference in their entireties as a part of the present application.

A computer readable xml file, entitled “Sequence Listing US 17-601125.xml,” created on or about Aug. 23, 2023, with a file size of 36,608 bytes contains the sequence listing for this application and is hereby incorporated by reference in its entirety.

### TECHNICAL FIELD

The present disclosure relates to a method for expression, water solubilization, and purification of proteins via a CSQ-tag (calsequestrin-tag) and provides a fusion protein comprising a CSQ-tag and a protein of interest, a nucleic acid including a nucleotide sequence coding for the fusion protein, an expression vector carrying the nucleic acid, a cell transformed with the expression vector, and a method for expressing, water-solubilizing, and purifying the protein of interest by using the CSQ tag, the present disclosure is expected to decrease production costs of protein substances and protein pharmaceuticals.

### BACKGROUND ART

With the technical advance of the genetic engineering and biology science, a great number of trials have recently been made to produce or obtain specific proteins on a mass scale and to apply the same to various industries and disease therapies. In this context, focus has been paid to the development of protein recombination, mass production, and purification technologies so as to acquire proteins of interest. For the most part, proteins of interest can be obtained through expression by culturing cells transformed with vectors which carry genes coding for the proteins of interest. In many cases, proteins can be expressed in eukaryotes, prokaryotes, etc. For some special cases, the expression may be achieved in transgenic plants or animals. By way of example, a trial may be made to express a protein of interest in a transgenic mammalian animal and to produce the same through the milk secreted therefrom. In this regard, the protein of interest can be separated and purified from the cell culture or milk.

When expression is carried out in plants, animals, or microorganisms which lack an additional strategy for obtaining a protein of interest through secretion, a process of extracting the protein from the storage organ or the inside of cells is required. However, the work of obtaining a protein of interest from transformed cells is not easy. Hence, a protein of interest is not produced in a native form, but now frequently in a recombinant form including a tag which is intended to facilitate protein purification. The use of tags in protein purification is a technique which is very highly efficient among various protein purification techniques. The tags available for the purpose are divided into peptide tags and protein tags. Peptide tags are composed of short amino acid sequences. Representative of the peptide tags is a His-tag (histidine-tag), especially, a hexahistidine tag (His6-tag). Since histidine peptides have specific chemical affinity for nickel, fusion proteins including corresponding tags can be purified at high purity by a nickel-containing column. A protein tag, which is designed to utilize a protein domain binding to a specific ingredient, includes the corresponding protein domain therein. A GST-tag (glutathione S-transferase-tag) is representative of protein tags. A GST tag can be purified at high purity by a column employing the GST substrate glutathione as a fixing mediator.

Leading to the present disclosure, intensive and thorough research, conducted by the present inventors, into the development of a novel tag facilitating the acquirement of a protein of interest, resulted in the finding that when fused with a CSQ (calsequestrin) tag, a protein of interest can be easily expressed, water-solubilized, and purified.

### RELATED ART DOCUMENT

Korean Patent Number 10-2014-0026781 A

### Technical Problem

An aspect of the present disclosure provides a fusion protein including a CSQ-tag and a protein of interest.

Another aspect of the present disclosure provides a nucleic acid including a nucleotide sequence coding for the fusion protein, and an expression vector carrying the nucleic acid. A schematic view of a recombinant expression vector according to the present disclosure is given in FIG. 1.

A further aspect of the present disclosure provides a cell transformed with the expression vector.

A still further aspect of the present disclosure provides a method for expressing and purifying a protein of interest, using a CSQ tag. The method for expressing and purifying a protein of interest according to the present disclosure is exemplified as seen in FIG. 2.

### Solution to Problem

The present disclosure pertains to a fusion protein including a CSQ tag and a protein of interest, wherein the CSQ tag acts to improve the expression and water solubility of the protein of interest.

The CSQ tag may be coded for by an amino acid sequence of SEQ ID NO: 1 or 2.

The CSQ tag may be encoded by a nucleotide sequence of SEQ ID NO: 3 or 4.

The CSQ tag and the protein of interest may be fused to each other via a hydrolase-cleavable peptide composed of an amino acid sequence selected from the group consisting of SEQ ID NOS: 5 to 8.

The protein of interest is selected from the group consisting of a polymer protein, a glycoprotein, a cytokine, a growth factor, a blood factor, a vaccine, a hormone, an enzyme, and an antibody. For example, the protein of interest may be selected from the group consisting of interleukin-2, blood clotting factor VII, blood clotting factor VIII, blood clotting factor IX, an immunoglobulin, horseradish peroxidase (HRP), a cytokine, α-interferon, β-interferon, γ-interferon, colony stimulating factor (GM-CSF), human fibronectin extra domain B (EBD), bone morphogenetic protein (BMP), fibroblast growth factor (FGF), vascular endothelial growth factor (VEGF), nerve growth factor (NGF), epidermal growth factor (EGF), insulin-like growth factor (IGF), trans-forming growth factor-α and -β (TGF-α and -β), brain-derived neurotrophic factor (BDNF), platelet-derived growth factor (PDGF), placental growth factor (P1GF), hepatocyte growth factor (HGF), fibroblast growth factor 1 and 2 (FGF-1 and -2), keratinocyte growth factor (KGF), glucagon-like peptide-1 (GLP-1), exendin, somatostatin, LHRH (luteinizing hormone-releasing hormone), adrenocorticotropic hormone, growth hormone-releasing hormone, oxytocin, thymosin alpha-1, corticotropin-releasing factor, calcitonin, bivalirudin, vasopressin, phospholipase-activating protein (PLAP), insulin, tumor necrosis factor (TNF), follicle-stimulating hormone, thyroid-stimulating hormone, antidiuretic hormone, pigmentary hormone, parathyroid hormone, luteinizing hormone, calcitonin gene-related peptide (CGRP), enkephalin, somatomedin, erythropoietin, hypothalamic releasing factor, prolactin, chorionic gonadotropin, tissue plasminogen activator, growth hormone releasing peptide (GHPR), thymic humoral factor (THF), asparaginase, arginase, arginine deiminase, adenosine deaminase, peroxidase dismutase, endotoxinase, catalase, chymotrypsin, lipase, uricase, adenosine diphosphatase, tyrosinase, bilirubin oxidase, glucose oxidase, glucosidase, galactosidase, glucocerebrosidase, and glucuronidase.

The protein of interest may be coded for by an amino acid sequence selected from the group consisting of SEQ ID NOS: 9 to 19.

Also, the present disclosure pertains to a nucleic acid including a nucleotide sequence encoding the fusion protein.

Also, the present disclosure pertains to an expression vector carrying the nucleic acid.

Also, the present disclosure pertains to a cell transformed with the expression vector.

The cell may be Escherichia coli, Bacillus subtilis, Bacillus thuringiensis, Salmonella typhimurium, Serratia marcescens, Pseudomonas spp. yeast, insect cells, CHO (Chinese hamster ovary) cells, W138, BHK, COS-7, 293, HepG2, 3T3, RIN, MDCK cells, or plant cells.

The present disclosure pertains to a method for expressing and purifying a protein of interest by using a CSQ tag, the method comprising the steps of constructing an expressing vector carrying a nucleic acid coding for the fusion protein:
B) transducing the expression vector into a host cell to obtain a transformant;
C) expressing the fusion protein including the CSQ tag and the protein of interest in the transformant;
D) precipitating the fusion protein including the CSQ tag and the protein of interest from the transformant with the aid of metal; and
E) separating the protein of interest from the fusion protein with a hydrolase.

The present disclosure also pertains to a method for expressing and purifying a protein of interest by using a CSQ tag, the method comprising the steps of constructing an expressing vector carrying a nucleic acid coding for the fusion protein:
B) transducing the expression vector into a host cell to obtain a transformant;
C) expressing the fusion protein including the CSQ tag and the protein of interest in the transformant;
D) precipitating the fusion protein including the CSQ tag and the protein of interest from the transformant with the aid of calcium; and
E) separating the protein of interest from the fusion protein with a hydrolase.

### Advantageous Effects

According to the method for expressing, water-solubilizing, and purifying a protein of interest by using CSQ tag of the present disclosure, a protein that is unlikely to express in an aqueous state or is likely to aggregate in an aqueous condition can be expressed in an aqueous state due to the high expression and water solubility of CSQ and can be separated and purified in a column-less manner by precipitation with metal. Thus, the present disclosure is expected to lower production costs of protein substances and protein pharmaceuticals.

### BRIEF DESCRIPTION OF DRAWINGS

FIG. 1 shows schematic diagrams of recombinant expression vectors according to the present disclosure.

FIG. 2 is a schematic view illustrating an expression and purification method for a protein of interest according to the present disclosure.

FIG. 3 shows data of cloning a gene coding for the protein of interest EDB into CSQ1TEV or CSQ1Thrombin vector according to Example 1.

FIG. 4 shows purification results of CSQ1TEV-EDB and CSQ1Thrombin-EDB proteins according to Example 1 in comparison with purification of EDB protein as fusions with the conventional commercially available tags His tag and GST. Although EDB14 protein can be expressed using His-tag or GST-tag, the protein of interest is not detected in a supernatant, but aggregates into an insoluble pellet. However, when expressed by using CSQ-tag, EDB14, 21, and 26 proteins, although aggregating in part into pellets, exist as soluble forms at a rate of about 30-50% in the supernatant (S). Thus, the data imply that it is possible to elute a protein of interest.

FIG. 5 shows results of digesting CSQ1TEV-EDB protein with TEV protease according to Example 1. After CSQ-tag and EDB protein were separated from each other by TEV protease, the application of calcium precipitates the separated CSQ-tag so that the protein of interest EDB can be easily purified.

FIG. 6 shows data of cloning a gene coding for the protein of interest EGF into CSQ1TEV or CSQ1Thrombin vector according to Example 2.

FIG. 7 shows data of purifying CSQ1Thrombin-EGF and CSQ1TEV-EGF proteins according to Example 2. EGF expressed in bacterial cells is known to exist in the form of pellets at a rate of 100%. However, the use of CSQ-tag is shown to make the protein soluble at a rate of 30-40%.

FIG. 8 shows data of digesting CSQ1Thrombin-EGF protein with thrombin protease according to Example 2.

FIG. 9 shows data of purifying CSQ1TEV-KGF1 protein according to Example 3.

FIG. 10 shows data of digesting CSQ1TEV-KGF1 with TEV protease according to Example 3.

FIG. 11 shows data of purifying CSQ1TEV-VEGF protein according to Example 3.

FIG. 12 shows data of digesting CSQ1TEV-VEGF protein with TEV protease according to Example 3.

FIG. 13 shows data of purifying CSQ1TEV-FGF2 protein according to Example 3.

FIG. 14 shows data of digesting CSQ1TEV-FGF2 protein with TEV protease according to Example 3.

FIG. 15 shows data of cloning genes coding for the proteins of interest BMP2 and TGFβ into TEVCSQ1 or ThrombinCSQ1 vector according to Example 4.

FIG. 16 shows data of purifying BMP2-TEVCSQ1 and BMP2-ThrombinCSQ1 proteins according to Example 4.

FIG. 17 shows data of digesting BMP2-TEVCSQ1 or BMP2-ThrombinCSQ1 protein with TEV protease or Thrombin protease according to Example 4.

FIG. 18 shows data of purifying TGFβ-TEVCSQ1 and TGFβ-ThrombinCSQ1 proteins according to Example 4.

FIG. 19 shows data of digesting TGFβ-TEVCSQ1 or TGFβ-ThrombinCSQ1 protein with TEV protease or Thrombin protease according to Example 4.

FIG. 20 shows data of purifying HRP-TEVCSQ1 protein according to Example 4.

FIG. 21 shows data of digesting HRP-TEVCSQ1 protein with TEV protease according to Example 4.

FIG. 22 shows data of purifying GLP1-TEVCSQ1 protein according to Example 4.

FIG. 23 shows data of digesting GLP1-TEVCSQ1 protein with TEV protease according to Example 4.

FIG. 24 shows data of cloning genes coding for the proteins of interest BMP2 and KGF1 into CSQ2TEV or CSQ2Thrombin vector according to Example 5.

FIG. 25 shows data of purifying CSQ2TEV-BMP2 and CSQ2Thrombin-BMP2 proteins according to Example 5.

FIG. 26 shows data of digesting CSQ2TEV-BMP2 or CSQ2Thrombin-BMP2 protein with TEV protease or Thrombin protease according to Example 5.

FIG. 27 shows data of purifying CSQ2TEV-KGF1 and CSQ2Thrombin-KGF1 proteins according to Example 5.

FIG. 28 shows data of digesting CSQ2TEV-KGF1 or CSQ2Thrombin-KGF1 protein with TEV protease or Thrombin protease according to Example 5.

FIG. 29 shows data of purifying HRP-TEVCSQ2 and HRP-ThrombinCSQ2 proteins according to Example 6.

FIG. 30 shows data of digesting HRP-TEVCSQ2 protein with TEV protease according to Example 6.

FIG. 31 shows data of purifying GLP1-TEVCSQ2 protein according to Example 6.

FIG. 32 shows data of digesting GLP1-TEVCSQ2 protein with TEV protease according to Example 6.

FIG. 33 shows data of purifying CSQ1-Ssp Dna-EGF protein according to Example 7.

FIG. 34 shows data of cleaving CSQ1-Ssp Dna-EGF protein with dithiothreitol (DTT) according to Example 7.

FIG. 35 shows data of purifying CSQ2-Ssp Dna-KGF1 protein according to Example 7.

FIG. 36 shows data of cleaving CSQ2-Ssp Dna-KGF1 protein with dithiothreitol (DTT) according to Example 7.

FIG. 37 shows data of purifying EGF-GyrA-CSQ1 protein according to Example 8.

FIG. 38 shows data of cleaving EGF-GyrA-CSQ1 protein with 20 mM Na-HEPES (pH 6.5) buffer according to Example 8.

FIG. 39 shows data of purifying BMP2-GyrA-CSQ2 protein according to Example 8.

FIG. 40 shows data of cleavage with 20 mM Na-HEPES (pH 6.5) buffer according to Example 8.

FIG. 41 shows data of purification of CSQ-BMP2 using various metals (SDS-PAGE)

FIG. 42 displays a histogram comparing the purity of CSQ-BMP2 obtained from

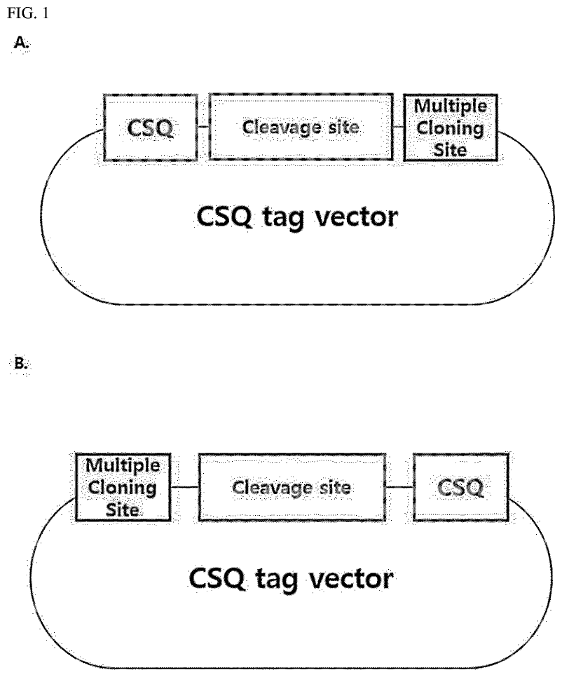

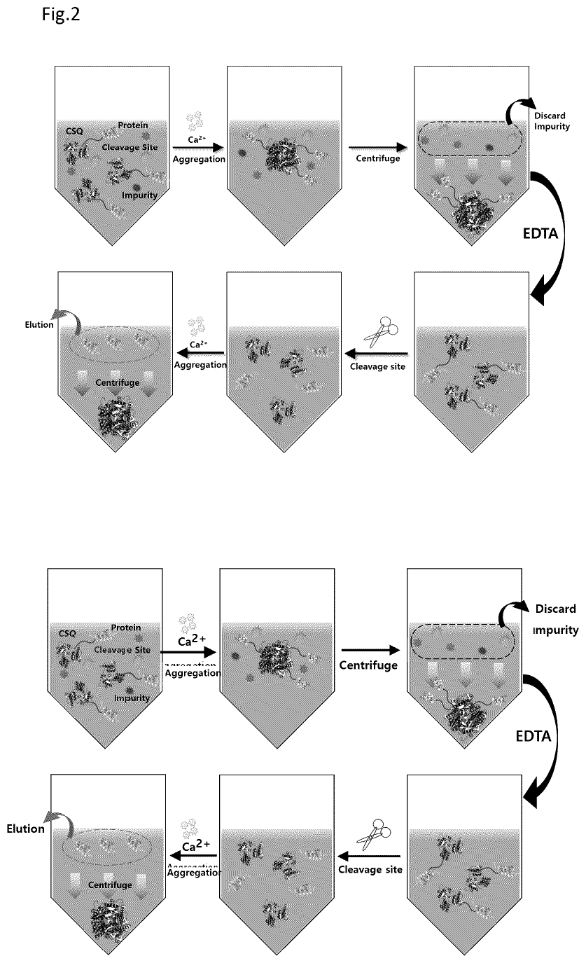

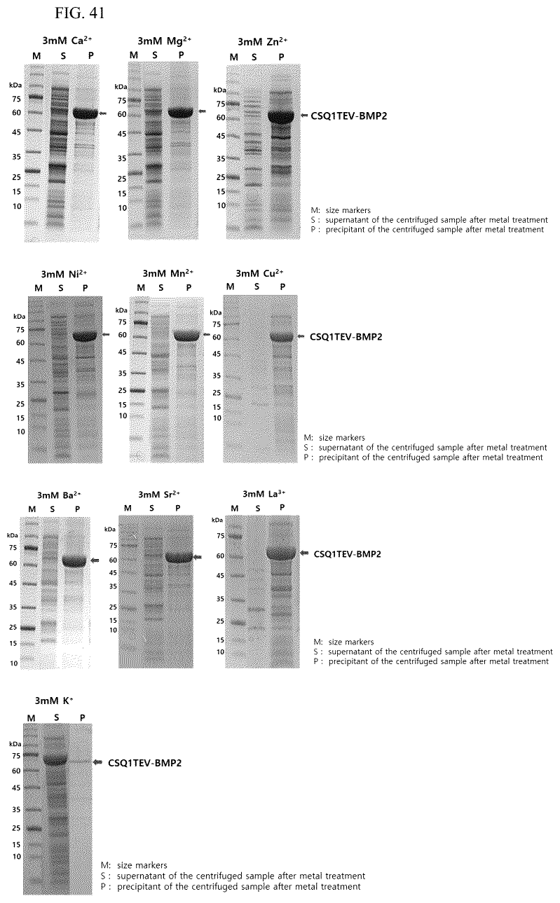

FIG. 41 using Fusion FX analysis equipment and Evolution-capt Edge software.

FIG. 43 shows the results of turbidity measurements of CSQ1TEV-BMP2 after reacting with various metal ions (Ca2+, Mg2+, Zn2+, Ni2+, Mn2+, Cu2+, Ba2+, Sr2+, La3+, K+).

### BEST MODE

The term “calsequestrin” or “CSQ”, as used herein, refers to a metal-binding protein that acts as a metal buffer within the sarcoplasmic reticulum and helps hold metal in the cisterna of the sarcoplasmic reticulum by binding metal after a muscle contraction, even though the contraction of metal in the sarcoplasmic reticulum is much higher than in the cytosol. Each molecule of calsequestrin can bind many metal ions (e.g., one molecule of calsequestrin has 40-50 metal-binding sites) and as such, exhibits a very high capability of storing metal. Calsequestrin exists as monomers at a low concentration of metal and forms a polymer and increases in size as metal concentration is increased. Herein, a “metal” may include metal and metal ion. Similarly, the term “calcium” may include Ca and Ca2+. In some embodiments, the metal may comprise at least one selected from the group consisting of Ca, Cu, Ra, Ba, Sr, Ca, Cd, Co, Cr, Cu, Be, Fe, Zn, Mg, Mn, Co, Ni, Al, Fe, Au, He, Cr, Si, V, Ga, In, La, Ce and ions thereof. In some embodiments, the metal may comprise Ca2+. In some embodiments, the metal may comprise Mg2+. In some embodiments, the metal may comprise Zn2+. In some embodiments, the metal may comprise Ni2+. In some embodiments, the metal may comprise Mn2+. In some embodiments, the metal may comprise Cu2+. In some embodiments, the metal may comprise Ba2+. In some embodiments, the metal may comprise Sr2+. In some embodiments, the metal may comprise La3+.

As used herein, the term “protein of interest” refers to a protein that is required to be obtained at high purity or in a large amount according to a specific purpose and is intended to encompass any native protein, variant protein, or novel recombinant protein, with not limitations thereto. A protein of interest may be a protein that is required at high purity or in a large amount for industrial, medical, or scientific reason, or other reasons and particularly, may be a recombinant protein for medical or academic studies and more particularly, may be selected from the group consisting of a polymer protein, a glycoprotein, a cytokine, a growth factor, a blood factor, a vaccine, a hormone, an enzyme, and an antibody. The protein of interest may be far more particularly an entirety or portion of a light or a heavy chain of an antibody and most particularly a light-chain variable region (VL) or a heavy-chain variable region (VH) of an antibody.

As used herein, the term “hydrolase-cleavable peptide” refers to a peptide that can be cleaved by a hydrolase. The hydrolase-cleavable peptide may be located between a CSQ tag and a protein of interest and is cleaved by a hydrolase to separate the CSQ tag and the protein of interest.

As used herein, the term “vector” is an expression vector that allows the expression of a protein of interest in a suitable host cell and refers to a nucleic acid construct including essential regulatory elements operably linked to express a nucleic acid insert. So long as it is usually used, any plasmid may be used as the vector. Examples of the plasmid include bacterial plasmids, phages, yeast plasmids, plant cell viruses, mammalian cell viruses, and other vectors. The vector may be constructed by manipulating vectors usually used in the art, such as plasmids (e.g., pSC101, pGV1106, pACYC177. Co1E1, pKT230, pME290, pBR322, pUC8/9, pUC6, pBD9, pHC79, pIJ61, pLAFR1, pHV14, pGEX series, pET series, and pUC19), phages (e.g., λgt4λ.B. λ-Charon. λΔz1. M13, etc.), or viruses (e.g., CMV. SV40, etc.).

As used herein, the term “transformation” means introduction of DNA into a host cell so that the DNA is replicable either as an extra-chromosomal element or by chromosomal integration and refers to artificial genetic alteration by introducing a foreign DNA into a host cell. Examples of the transformation available in the present disclosure include, but are not limited to, CaCl2 precipitation, a Hanahan method, which employs DMSO (dimethyl sulfoxide) as a reducing material in combination with CaCl2 precipitation so as to improve the efficiency, electroporation, calcium phosphate precipitation, protoplast fusion, vortexing in the presence of silicon carbide fibers, agrobacterium-mediated transformation, PEG-mediated transformation, dextran sulfate-, lipofectamine-, and desiccation/inhibition-mediated transformation.

As used herein, the term “host cell” refers to a cell that allows a guest, such as a different microbe or gene to be introduced thereinto and supplies nutrients to the guest. In this context, the host cell means a cell transformed with a vector wherein the vector exhibits various genetic or molecular influences. So long as it allows the vector of the present disclosure to be cloned and expressed stably and continuously therein, any host cells known in the art may be employed in the present disclosure. Examples of available prokaryotic host cells include E. coli Rosetta. E. coli JM109. E. coli BL21. E. coli RR1. E. coli LE392. E. coli B. E. coli X 1776. E. coli W3110, Bacillus spp. such as Bacillus subtilis and Bacillus thuringiensis, and gut bacteria such as Salmonella typhimurium, Serratia marcescens, and various Pseudomonas spp. As eukaryotic host cells into which the vector of the present disclosure is transformed, yeast (Saccharomyce cerevisiae), insect cells, human cells (e.g., CHO (Chinese hamster ovary) cells, W138, BHK, COS-7, 293, HepG2, 3T3, RIN, and MDCK cells), and plant cells may be used.

In one aspect, the present disclosure provides a fusion protein comprising a CSQ tag and a protein of interest.

In the fusion protein according to the present disclosure, the CSQ tag includes CSQ1 or CSQ2. In some embodiments, the CSQ tag includes CSQ1. In some embodiments, the CSQ tag includes CSQ2

In the fusion protein according to the present disclosure, the CSQ1 and the CSQ2 may have amino acid sequences having at least 80, 85, 90, 91, 92, 93, 94, 95, 96, 97, 98, 99 or 100% sequence identities to SEQ ID NOS: 1 and 2, respectively. The CSQ1 or the amino acid sequence of SEQ ID NO: 1 may be encoded by a sequence having at least 80, 85, 90, 91, 92, 93, 94, 95, 96, 97, 98, 99 or 100% sequence identity to the nucleotide sequence of SEQ ID NO: 3. The CSQ2 or the amino acid sequence of SEQ ID NO: 2 may be encoded by a sequence having at least 80, 85, 90, 91, 92, 93, 94, 95, 96, 97, 98, 99 or 100% sequence identity to the nucleotide sequence of SEQ ID NO: 4.

In the fusion protein according to the present disclosure, the CSQ tag and the protein of interest may be fused to each other via a hydrolase-cleavable peptide composed of an amino acid sequence selected from the group consisting of sequences having at least 80, 85, 90, 91, 92, 93, 94, 95, 96, 97, 98, 99 or 100% sequence identities to SEQ ID NOS: 5 to 8.

In the fusion protein according to the present disclosure, the protein of interest is selected from the group consisting of a polymer protein, a glycoprotein, a cytokine, a growth factor, a blood factor, a vaccine, a hormone, an enzyme, and an antibody. For example, the protein of interest may be selected from the group consisting of interleukin-2, blood clotting factor VII, blood clotting factor VIII, blood clotting factor IX, an immunoglobulin, horseradish peroxidase (HRP), a cytokine, α-interferon, β-interferon, γ-interferon, colony stimulating factor (GM-CSF), human fibronectin extra domain B (EBD), bone morphogenetic protein (BMP), fibroblast growth factor (FGF), vascular endothelial growth factor (VEGF), nerve growth factor (NGF), epidermal growth factor (EGF), insulin-like growth factor (IGF), trans-forming growth factor-α and -β (TGF-α and -β), brain-derived neurotrophic factor (BDNF), platelet-derived growth factor (PDGF), placental growth factor (P1GF), hepatocyte growth factor (HGF), fibroblast growth factor 1 and 2 (FGF-1 and -2), keratinocyte growth factor (KGF), glucagon-like peptide-1 (GLP-1), exendin, somatostatin, LHRH (luteinizing hormone-releasing hormone), adrenocorticotropic hormone, growth hormone-releasing hormone, oxytocin, thymosin alpha-1, corticotropin-releasing factor, calcitonin, bivalirudin, vasopressin, phospholipase-activating protein (PLAP), insulin, tumor necrosis factor (TNF), follicle-stimulating hormone, thyroid-stimulating hormone, antidiuretic hormone, pigmentary hormone, parathyroid hormone, luteinizing hormone, calcitonin gene-related peptide (CGRP), enkephalin, somatomedin, erythropoietin, hypothalamic releasing factor, prolactin, chorionic gonadotropin, tissue plasminogen activator, growth hormone releasing peptide (GHPR), thymic humoral factor (THF), asparaginase, arginase, arginine deiminase, adenosine deaminase, peroxidase dismutase, endotoxinase, catalase, chymotrypsin, lipase, uricase, adenosine diphosphatase, tyrosinase, bilirubin oxidase, glucose oxidase, glucosidase, galactosidase, glucocerebrosidase, and glucuronidase.

In the fusion protein according to the present disclosure, the protein of interest may be coded for by an amino acid sequence selected from the group consisting of SEQ ID NOS: 9 to 19.

In one aspect, the present disclosure provides a nucleic acid coding for a fusion protein including a CSQ tag and a protein of interest, and an expression vector carrying the nucleic acid.

In the nucleic acid or expression vector according to the present disclosure, the CSQ tag may be coded for by an amino acid sequence of SEQ ID NO: 1 or 2. The amino acid sequence of SEQ ID NO: 1 may be encoded by the nucleotide sequence of SEQ ID NO: 3 and the amino acid sequence of SEQ ID NO: 2 may be encoded by the nucleotide sequence of SEQ ID NO: 4.

In the nucleic acid or expression vector according to the present disclosure, the CSQ tag and the protein of interest may be fused to each other via a hydrolase-cleavable peptide composed of an amino acid sequence selected from the group consisting of SEQ ID NOS: 5 to 8.

In the nucleic acid or expression vector according to the present disclosure, the protein of interest is selected from the group consisting of a polymer protein, a glycoprotein, a cytokine, a growth factor, a blood factor, a vaccine, a hormone, an enzyme, and an antibody. For example, the protein of interest may be selected from the group consisting of interleukin-2, blood clotting factor VII, blood clotting factor VIII, blood clotting factor IX, an immunoglobulin, horseradish peroxidase (HRP), a cytokine, α-interferon, β-interferon, γ-interferon, colony stimulating factor (GM-CSF), human fibronectin extra domain B (EBD), bone morphogenetic protein (BMP), fibroblast growth factor (FGF), vascular endothelial growth factor (VEGF), nerve growth factor (NGF), epidermal growth factor (EGF), insulin-like growth factor (IGF), trans-forming growth factor-α and -β (TGF-α and -β), brain-derived neurotrophic factor (BDNF), platelet-derived growth factor (PDGF), placental growth factor (P1GF), hepatocyte growth factor (HGF), fibroblast growth factor 1 and 2 (FGF-1 and -2), keratinocyte growth factor (KGF), glucagon-like peptide-1 (GLP-1), exendin, somatostatin, LHRH (luteinizing hormone-releasing hormone), adrenocorticotropic hormone, growth hormone-releasing hormone, oxytocin, thymosin alpha-1, corticotropin-releasing factor, calcitonin, bivalirudin, vasopressin, phospholipase-activating protein (PLAP), insulin, tumor necrosis factor (TNF), follicle-stimulating hormone, thyroid-stimulating hormone, antidiuretic hormone, pigmentary hormone, parathyroid hormone, luteinizing hormone, calcitonin gene-related peptide (CGRP), enkephalin, somatomedin, erythropoietin, hypothalamic releasing factor, prolactin, chorionic gonadotropin, tissue plasminogen activator, growth hormone releasing peptide (GHPR), thymic humoral factor (THF), asparaginase, arginase, arginine deiminase, adenosine deaminase, peroxidase dismutase, endotoxinase, catalase, chymotrypsin, lipase, uricase, adenosine diphosphatase, tyrosinase, bilirubin oxidase, glucose oxidase, glucosidase, galactosidase, glucocerebrosidase, and glucuronidase.

In the nucleic acid or expression vector according to the present disclosure, the protein of interest may be coded for by an amino acid sequence selected from the group consisting of SEQ ID NOS: 9 to 19.

In one aspect, the present disclosure provides a cell transformed with an expression vector carrying a nucleic acid coding for a fusion protein including a CSQ tag and a protein of interest.

In the transformed cell according to the present disclosure, the CSQ tag may be coded for by an amino acid sequence of SEQ ID NO: 1 or 2. The amino acid sequence of SEQ ID NO: 1 may be encoded by the nucleotide sequence of SEQ ID NO: 3 and the amino acid sequence of SEQ ID NO: 2 may be encoded by the nucleotide sequence of SEQ ID NO: 4.

In the transformed cell according to the present disclosure, the CSQ tag and the protein of interest may be fused to each other via a hydrolase-cleavable peptide composed of an amino acid sequence selected from the group consisting of SEQ ID NOS: 5 to 8.

In the transformed cell according to the present disclosure, the protein of interest is selected from the group consisting of a polymer protein, a glycoprotein, a cytokine, a growth factor, a blood factor, a vaccine, a hormone, an enzyme, and an antibody. For example, the protein of interest may be selected from the group consisting of interleukin-2, blood clotting factor VII, blood clotting factor VIII, blood clotting factor IX, an immunoglobulin, horseradish peroxidase (HRP), a cytokine, α-interferon. β-interferon, γ-interferon, colony stimulating factor (GM-CSF), human fibronectin extra domain B (EBD), bone morphogenetic protein (BMP), fibroblast growth factor (FGF), vascular endothelial growth factor (VEGF), nerve growth factor (NGF), epidermal growth factor (EGF), insulin-like growth factor (IGF), trans-forming growth factor-α and -β (TGF-α and -β), brain-derived neurotrophic factor (BDNF), platelet-derived growth factor (PDGF), placental growth factor (P1GF), hepatocyte growth factor (HGF), fibroblast growth factor 1 and 2 (FGF-1 and -2), keratinocyte growth factor (KGF), glucagon-like peptide-1 (GLP-1), exendin, somatostatin. LHRH (luteinizing hormone-releasing hormone), adrenocorticotropic hormone, growth hormone-releasing hormone, oxytocin, thymosin alpha-1, corticotropin-releasing factor, calcitonin, bivalirudin, vasopressin, phospholipase-activating protein (PLAP), insulin, tumor necrosis factor (TNF), follicle-stimulating hormone, thyroid-stimulating hormone, antidiuretic hormone, pigmentary hormone, parathyroid hormone, luteinizing hormone, calcitonin gene-related peptide (CGRP), enkephalin, somatomedin, erythropoietin, hypothalamic releasing factor, prolactin, chorionic gonadotropin, tissue plasminogen activator, growth hormone releasing peptide (GHPR), thymic humoral factor (THF), asparaginase, arginase, arginine deiminase, adenosine deaminase, peroxidase dismutase, endotoxinase, catalase, chymotrypsin, lipase, uricase, adenosine diphosphatase, tyrosinase, bilirubin oxidase, glucose oxidase, glucosidase, galactosidase, glucocerebrosidase, and glucuronidase.

In the transformed cell according to the present disclosure, the protein of interest may be coded for by an amino acid sequence selected from the group consisting of SEQ ID NOS: 9 to 19.

In the transformed cell according to the present disclosure, the cell may be Escherichia coli, Bacillus subtilis, Bacillus thuringiensis, Salmonella typhimurium, Serratia marcescens, Pseudomonas spp. yeast, insect cells, CHO (Chinese hamster ovary) cells, W138, BHK, COS-7, 293, HepG2, 3T3, RIN, MDCK cells, or plant cells.

In one aspect, the present disclosure provides a method for expressing and purifying a protein of interest by using a CSQ tag, the method comprising the steps of:
A) constructing an expressing vector carrying a nucleic acid coding for a fusion protein wherein the fusion protein includes a CSQ tag and a protein of interest;
B) transducing the expression vector into a host cell to obtain a transformant;
C) expressing the fusion protein including the CSQ tag and the protein of interest in the transformant;
D) precipitating the fusion protein including the CSQ tag and the protein of interest from the transformant with the aid of metal; and
E) separating the protein of interest from the fusion protein with a hydrolase.

In some embodiments, the metal may comprise at least one selected from the group consisting of Ca, Cu, Ra, Ba, Sr, Ca, Cd, Co, Cr, Cu, Be, Fe, Zn, Mg, Mn, Co, Ni, Al, Fe, Au, He, Cr, Si, V, Ga, In, La, Ce and ions thereof. In some embodiments, the metal may comprise Ca2+. In some embodiments, the metal may comprise Mg2+. In some embodiments, the metal may comprise Zn2+. In some embodiments, the metal may comprise Ni2+. In some embodiments, the metal may comprise Mn2+. In some embodiments, the metal may comprise Cu2+. In some embodiments, the metal may comprise Ba2+. In some embodiments, the metal may comprise Sr2+. In some embodiments, the metal may comprise La3+.

In the method for expressing and purifying a protein of interest according to the present disclosure, the CSQ tag may be coded for by an amino acid sequence of SEQ ID NO: 1 or 2. The amino acid sequence of SEQ ID NO: 1 may be encoded by the nucleotide sequence of SEQ ID NO: 3 and the amino acid sequence of SEQ ID NO: 2 may be encoded by the nucleotide sequence of SEQ ID NO: 4.

In the method for expressing and purifying a protein of interest according to the present disclosure, the CSQ tag and the protein of interest may be fused to each other via a hydrolase-cleavable peptide composed of an amino acid sequence selected from the group consisting of SEQ ID NOS: 5 to 8.

In the method for expressing and purifying a protein of interest according to the present disclosure, the protein of interest is selected from the group consisting of a polymer protein, a glycoprotein, a cytokine, a growth factor, a blood factor, a vaccine, a hormone, an enzyme, and an antibody. For example, the protein of interest may be selected from the group consisting of interleukin-2, blood clotting factor VII, blood clotting factor VIII, blood clotting factor IX, an immunoglobulin, horseradish peroxidase (HRP), a cytokine, α-interferon, β-interferon, γ-interferon, colony stimulating factor (GM-CSF), human fibronectin extra domain B (EBD), bone morphogenetic protein (BMP), fibroblast growth factor (FGF), vascular endothelial growth factor (VEGF), nerve growth factor (NGF), epidermal growth factor (EGF), insulin-like growth factor (IGF), trans-forming growth factor-α and -β (TGF-α and -β), brain-derived neurotrophic factor (BDNF), platelet-derived growth factor (PDGF), placental growth factor (P1GF), hepatocyte growth factor (HGF), fibroblast growth factor 1 and 2 (FGF-1 and -2), keratinocyte growth factor (KGF), glucagon-like peptide-1 (GLP-1), exendin, somatostatin, LHRH (luteinizing hormone-releasing hormone), adrenocorticotropic hormone, growth hormone-releasing hormone, oxytocin, thymosin alpha-1, corticotropin-releasing factor, calcitonin, bivalirudin, vasopressin, phospholipase-activating protein (PLAP), insulin, tumor necrosis factor (TNF), follicle-stimulating hormone, thyroid-stimulating hormone, antidiuretic hormone, pigmentary hormone, parathyroid hormone, luteinizing hormone, calcitonin gene-related peptide (CGRP), enkephalin, somatomedin, erythropoietin, hypothalamic releasing factor, prolactin, chorionic gonadotropin, tissue plasminogen activator, growth hormone releasing peptide (GHPR), thymic humoral factor (THF), asparaginase, arginase, arginine deiminase, adenosine deaminase, peroxidase dismutase, endotoxinase, catalase, chymotrypsin, lipase, uricase, adenosine diphosphatase, tyrosinase, bilirubin oxidase, glucose oxidase, glucosidase, galactosidase, glucocerebrosidase, and glucuronidase.

In the method for expressing and purifying a protein of interest according to the present disclosure, the protein of interest may be coded for by an amino acid sequence selected from the group consisting of SEQ ID NOS: 9 to 19.

A better understanding of the present disclosure may be obtained via the following examples which are set forth to illustrate, but are not to be construed as illustrating the present disclosure.

### Examples

### Example 1: Separation of EDB Using CSQ-Tag

1-1. Cloning of EDB into CSQ1TEV or CSQ1Thrombin Vector

In order to clone EDB into CSQ1TEV or CSQ1Thrombin vector, the following oligonucleotides EDB-F1 and EDB-B1 were synthesized (Bioneer Corporation, Daejeon, Korea). For comparison, a fusion protein of a well-known His tag or GST tag and EDB was prepared.

(SEQ ID NO: 20)
5′-AATGGATCCGAGGTGCCCCAACTCACTGAC-3′
(SEQ ID NO: 21)
5′-ATTCTCGAGTTACGTTTGTTGTGTCAGTGTAGTAGG-3′

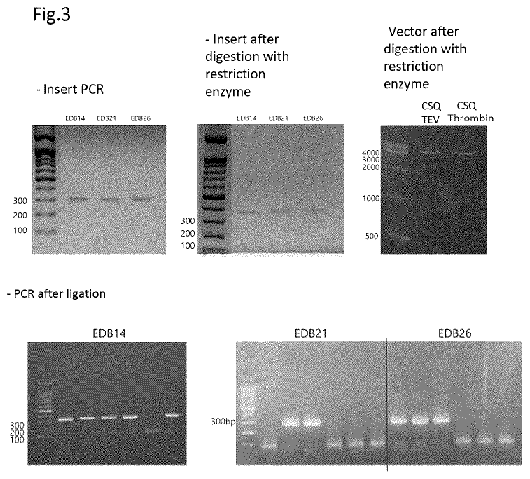

For use in amplification, EDB-F1 20 pmol, EDB-B1 20 pmol, PCR PreMix 4 μl (Elpisbio, Daejeon, Korea) and a template 10 μg were mixed and added with distilled water to form a final volume of 20 μl. The mixture was subjected to PCR (95° C., for 5 min, 30 cycles of: 95° C., 30 sec: 42° C., 30 sec; and 72° C., 45 sec, and 72° C., 5 min), followed by purification (PCR purification kit, GeneAll, Seoul, Korea) to obtain EDB14 (SEQ ID NO: 9), EDB21 (SEQ ID NO: 10), and EDB26 (SEQ ID NO: 11) genes. In order to insert the EDB genes into a CSQ1TEV or CSQ1Thrombin vector, the CSQ1TEV or CSQ1Thrombin vector and the insert DNAs were treated with restriction enzymes. About 1 μg of the insert DNA was incubated overnight with BamHI (New England Biolabs (NEB, Ipswich) and Xhol (NEB, Ipswich), followed by purifying the DNA digests through a PCR purification kit. In addition, about 40 μg of the CSQ1TEV or CSQ1Thrombin vector was incubated for 3 hours with CIAP (Calf Intestinal Alkaline Phosphatase) (NEB, Ipswich), followed by purification with a PCR purification kit. The DNA insert was ligated to the CSQTEV or CSQ1Thrombin vector at room temperature for 3 hours in the presence of T4 DNA ligase (Bioneer Corporation, Daejeon, Korea) (FIG. 3).

Next, transformation was made of the DNA resulting from ligating the EDB insert to the CSQ1TEV or CSQ1Thrombin vector. After being thawed on ice, 100 μl of DH5a competent cells was mixed and reacted for 30 min with 2 μl of the ligate solution. Heat shock was performed at 42° C., for 1 min on the reaction mixture which was then added with 200 μl of SOC medium and cultured at 37° C. for 30 min before being spread on plates. Of the colonies thus formed on the plate, six colonies were randomly picked up. The inserts were identified by PCR and DNA electrophoresis. The cloned colonies were committed to sequencing in Bioneer Corporation.

For comparison, a fusion protein of His tag or GST tag and EDB was cloned into pBT7-N-His and pBT7-N-GST vector provided from Bioneer Corporation in the same manner as described above.

### 1-2. Purification of CSQ1TEV-EDB. CSQ1Thrombin-EDB. His-EDB, and GST-EDB

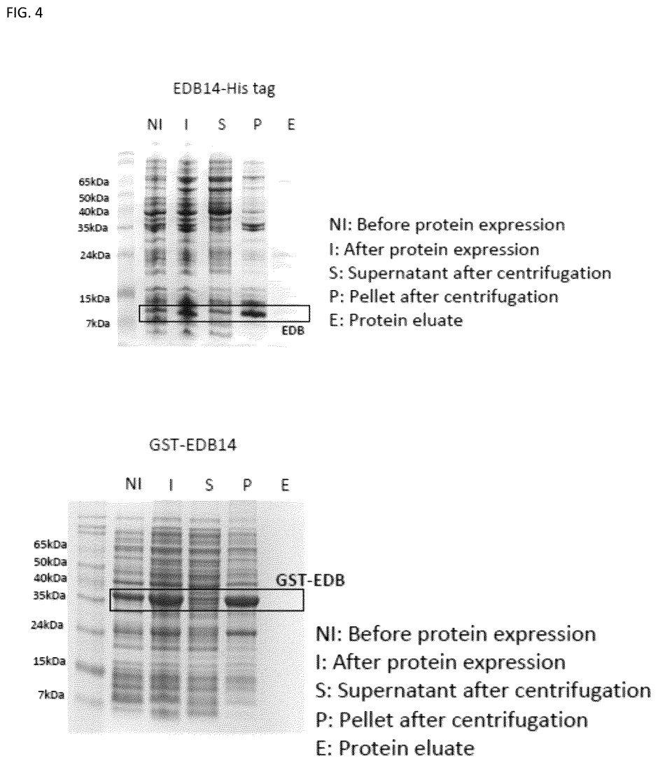

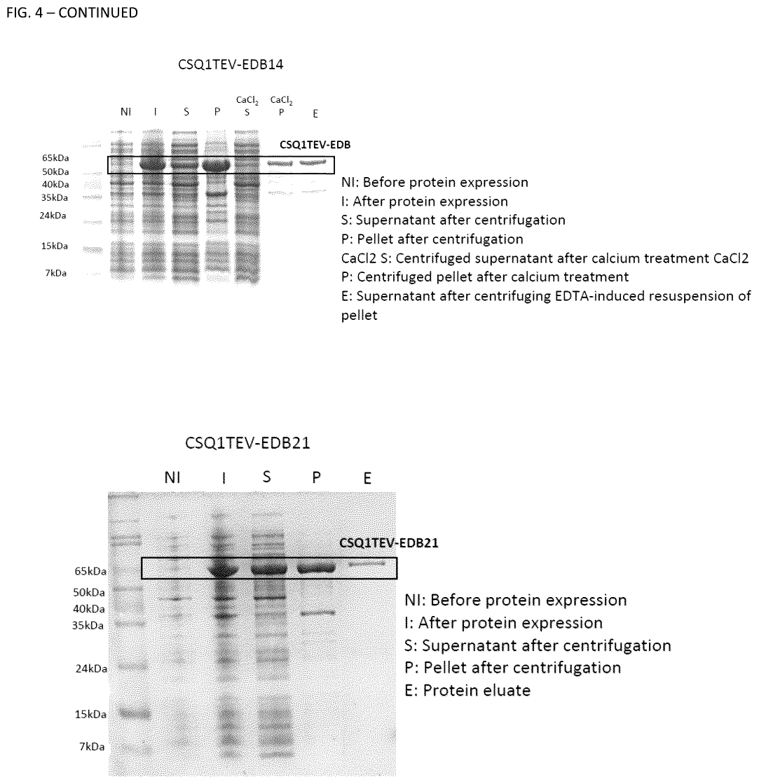

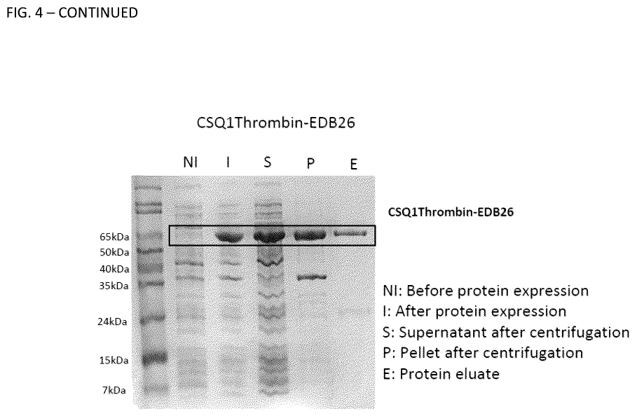

The DNAs sequenced in Bioneer Corporation were all transformed into BL21 cells which were then spread on agar plates containing ampicillin. Colonies grown on the agar plates were each inoculated into 5 ml of LB broth containing ampicillin (50 μg/ml) and cultured overnight at 37° C., while stirring at 200 rpm. The cell culture was mixed at a ratio of 1:1 with glycerol to give a 1-ml stock and stored at −80° C., in a deep-freezer. The stock was inoculated in an amount of 100 μl into 20 ml of LB broth containing ampicillin (50 μg/ml) and cultured overnight at 37° C., while stirring at 200 rpm. The culture was transferred to 400 ml of LB broth containing ampicillin (50 μg/ml) and incubated until OD=0.7. Then, additional incubation was carried out overnight at 18° C., in the presence of 1 mM isopropyl-β-D-thiogalatopyranoside (IPTG) while stirring at 200 rpm. After centrifugation at 4° C., and 4000 rpm for 20 min, the supernatant was discarded and the cell pellet was suspended in a lysis buffer (20 mM Tris (pH 8.0). 300 mM NaCl, and 10 mM imidazole). The suspension was stored overnight at −80° C., and then completely thawed, followed by adding a solution of 10 mg of PMSF (phenyl methane sulfonyl fluoride) in 1 ml of DMSO to 1 ml of the thawed E. coli. The E. coli was lysed using a sonicator, followed by centrifugation at 4° C., and 13.000 rpm for 1 hour. The supernatant was incubated with 20 mM CaCl₂) at 4° C., for 1 hour and then centrifuged at 5000 rpm for 30 min. After removal of the supernatant, the pellet thus formed was suspended in EDTA to obtain CSQ1TEV-EDB and CSQ1Thrombin-EDB proteins (FIG. 4).

His-EDB was expressed and purified as follows. The pBT7-N-His vector having EDB14 cloned thereinto was transformed into BL21 cells which were then spread on agar plates containing ampicillin. Colonies grown on the agar plates were each inoculated into 5 ml of LB broth containing ampicillin (50 μg/ml) and cultured overnight at 37° C., while stirring at 220 rpm. The cell culture was mixed at a ratio of 1:1 with glycerol to give a 1-ml stock and stored at −80° C., in a deep-freezer. The stock was inoculated in an amount of 100 μl into 20 ml of LB broth containing ampicillin (50 μg/ml) and cultured overnight at 37° C., while stirring at 220 rpm. The culture was transferred to 400 ml of LB broth containing ampicillin (50 μg/ml) and incubated until OD=0.7. Then, additional incubation was carried out overnight at 37° C., in the presence of 1 mM isopropyl-β-D-thiogalatopyranoside (IPTG) while stirring at 220 rpm. After centrifugation at 4° C., and 4000 xg for 10 min, the supernatant was discarded and the cell pellet was suspended in a lysis buffer (50 mM sodium phosphate (pH 8.0). 300 mM NaCl and 10 mM imidazole). The suspension was stored overnight at −80° C., and then completely thawed, followed by adding a solution of 10 mg of PMSF (phenyl methane sulfonyl fluoride) in 1 ml of isopropanol to 1 ml of the thawed E. coli.

The E. coli was lysed using a sonicator, followed by centrifugation at 4° C., and 13,000 rpm for 1 hour. The supernatant was applied to a Ni-NTA affinity resin (Elposbio, Daejeon, Korea) which was previously washed with distilled water and a lysis buffer. Then, the resin was washed with 600 ml of a washing buffer (50 mM sodium phosphate (pH 8.0), 300 mM NaCl, and 20 mM imidazole), followed by elution with a butter (50 mM sodium phosphate (pH 8.0), 300 mM NaCl, and 250 mM imidazole) to acquire N-terminal His-tag EDB14 protein (FIG. 4).

GST-EDB was expressed and purified as follows. The pBT7-N-GST vector having EDB14 cloned thereinto was transformed into BL21 cells which were then spread on agar plates containing ampicillin. Colonies grown on the agar plates were each inoculated into 5 ml of LB broth containing ampicillin (50 μg/ml) and cultured overnight at 37° C., while stirring at 220 rpm. The cell culture was mixed at a ratio of 1:1 with glycerol to give a 1-ml stock and stored at −80° C., in a deep-freezer. The stock was inoculated in an amount of 100 μl into 20 ml of LB broth containing ampicillin (50 μg/ml) and cultured overnight at 37° C., while stirring at 220 rpm. The culture was transferred to 400 ml of LB broth containing ampicillin (50 μg/ml) and incubated until OD=0.7. Then, additional incubation was carried out overnight at 37° C., in the presence of 1 mM isopropyl-β-D-thiogalatopyranoside (IPTG) while stirring at 220 rpm. After centrifugation at 4° C., and 4000 xg for 10 min, the supernatant was discarded and the cell pellet was suspended in a lysis buffer (50 mM Tris(pH 7.5), 150 mM NaCl and 0.05% NP-40). The suspension was stored overnight at −80° C., and then completely thawed, followed by adding a solution of 10 mg of PMSF (phenyl methane sulfonyl fluoride) in 1 ml of isopropanol to 1 ml of the thawed E. coli.

The E. coli was lysed using a sonicator, followed by centrifugation at 4° C., and 13,000 rpm for 1 hour. The supernatant was applied to the GST.Bind Agarose Resin (Elposbio, Daejeon, Korea) which was previously washed with distilled water and a lysis buffer. Then, the resin was washed with 600 ml of a washing buffer (50 mM Tris-Cl (pH 8.0), 150 mM NaCl, and 0.1 mM EDTA), followed by elution with a (50 mM Tris-Cl (pH 8.0), 150 mM NaCl, 0.1 mM EDTA, and 10 mM Reduced(Free) glutathione) to acquire N-terminal GST-tag EDB14 protein (FIG. 4).

As a result, it was observed that EDB14 protein cannot be detected in the supernatant when using His-tag or GST-tag, but can be solubilized and detected in the supernatant when using CSQ-tag.

### 1-3. EDB Isolation Using TEV Protease

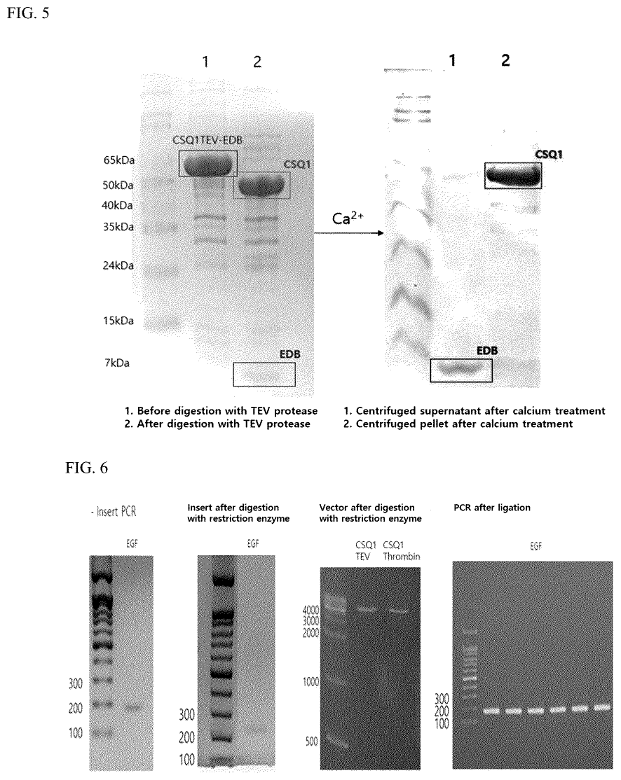

In order to isolate only EDB from CSQ1TEV-EDB protein, the recombinant protein was incubated overnight at 30° C., with 5 μl of TEV protease (Genscript, USA). The resulting digests were run by SDS-PAGE (FIG. 5). As a result, it was observed that the CSQ-tag and the EDB fusion protein can be easily segregated using TEV protease after precipitation with calcium.

### Example 2: Separation of EGF Using CSQ-Tag

2-1. Cloning of EGF into CSQ1TEV or CSQ1Thrombin Vector

In order to clone EDB into CSQ1TEV or CSQ1Thrombin vector, the following oligonucleotides EGF-F1 and EGF-B1 were synthesized (Bioneer Corporation, Daejeon, Korea)

(SEQ ID NO: 22)
5′-AATGGATCCAACTCTGATAGCGAATGCCCG-3′
(SEQ ID NO: 23)
5′-ATTCTCGAGTTAACGCAGTTCCCACCATTT-3′

For use in amplification, EGF-F1 20 pmol, EGF-B1 20 pmol, PCR PreMix 4 μl (Elpisbio, Daejeon, Korea) and a template 10 μg were mixed and added with distilled water to form a final volume of 20 μl. The mixture was subjected to PCR (95° C., for 5 min, 30 cycles of: 95° C., 30 sec: 42° C., 30 sec; and 72° C., 45 sec, and 72° C., 5 min), followed by purification (PCR purification kit, GeneAll, Seoul, Korea) to obtain a EGF gene (SEQ ID NO: 12). In order to insert the EGF gene into a CSQ1TEV or CSQ1Thrombin vector, the CSQ1TEV or CSQ1Thrombin vector and the insert DNAs were treated with restriction enzymes. About 1 μg of the insert DNA was incubated overnight with BamHI (New England Biolabs (NEB, Ipswich) and Xhol (NEB, Ipswich), followed by purifying the DNA digests through a PCR purification kit. In addition, about 40 μg of the CSQ1TEV or CSQ1Thrombin vector was incubated for 3 hours with CIAP (Calf Intestinal Alkaline Phosphatase) (NEB, Ipswich), followed by purification with a PCR purification kit. The DNA insert was ligated to the CSQTEV or CSQ1Thrombin vector at room temperature for 3 hours in the presence of T4 DNA ligase (Bioneer Corporation, Daejeon, Korea) (FIG. 6).

Next, transformation was made of the DNA resulting from ligating the EGF insert to the CSQ1TEV or CSQ1Thrombin vector. After being thawed on ice, 100 μl of DH5a competent cells was mixed and reacted for 30 min with 2 μl of the ligate solution. Heat shock was performed at 42° C., for 1 min on the reaction mixture which was then added with 200 μl of SOC medium and cultured at 37° C. for 30 min before being spread on plates. Of the colonies thus formed on the plates, six colonies were randomly picked up. The inserts were identified by PCR and DNA electrophoresis. The cloned colonies were committed to sequencing in Bioneer Corporation.

### 2-2. Purification of CSQ1Thrombin-EGF or CSQ1TEV-EGF

The DNA sequenced in Bioneer Corporation was transformed into BL21 cells which were then spread on agar plates containing ampicillin. Colonies grown on the agar plates were each inoculated into 5 ml of LB broth containing ampicillin (50 μg/ml) and cultured overnight at 37° C., while stirring at 200 rpm. The cell culture was mixed at a ratio of 1:1 with glycerol to give a 1-ml stock and stored at −80° C., in a deep-freezer. The stock was inoculated in an amount of 100 μl into 20 ml of LB broth containing ampicillin (50 μg/ml) and cultured overnight at 37° C., while stirring at 200 rpm. The culture was transferred to 400 ml of LB broth containing ampicillin (50 μg/ml) and incubated until OD=0.7. Then, additional incubation was carried out overnight at 18° C., in the presence of 1 mM isopropyl-β-D-thiogalatopyranoside (IPTG) while stirring at 200 rpm. After centrifugation at 4° C., and 4000 rpm for 20 min, the supernatant was discarded and the cell pellet was suspended in a lysis buffer (20 mM Tris (pH 8.0), 300 mM NaCl, and 10 mM imidazole). The suspension was stored overnight at −80° C., and then completely thawed, followed by adding a solution of 10 mg of PMSF (phenyl methane sulfonyl fluoride) in 1 ml of DMSO to 1 ml of the thawed E. coli. The E. coli was lysed using a sonicator before centrifugation at 4° C., and 13,000 rpm for 1 hour.

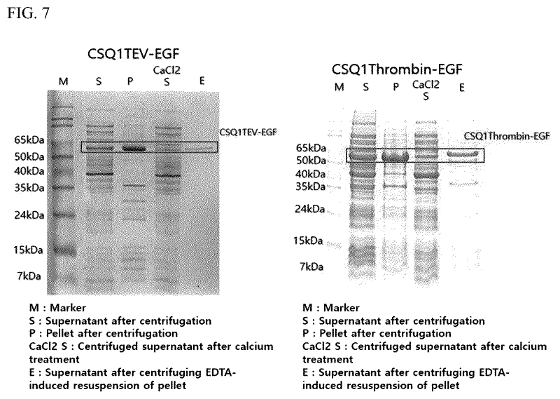

The supernatant was incubated with 20 mM CaCl₂) at 4° C. for 1 hour and then centrifuged at 5000 rpm for 30 min. After removal of the supernatant, the pellet thus formed was suspended in EDTA to obtain CSQ1Thrombin-EGF and CSQ1TEV-EGF proteins (FIG. 7).

### 2-3. EGF Isolation Using Thrombin Protease

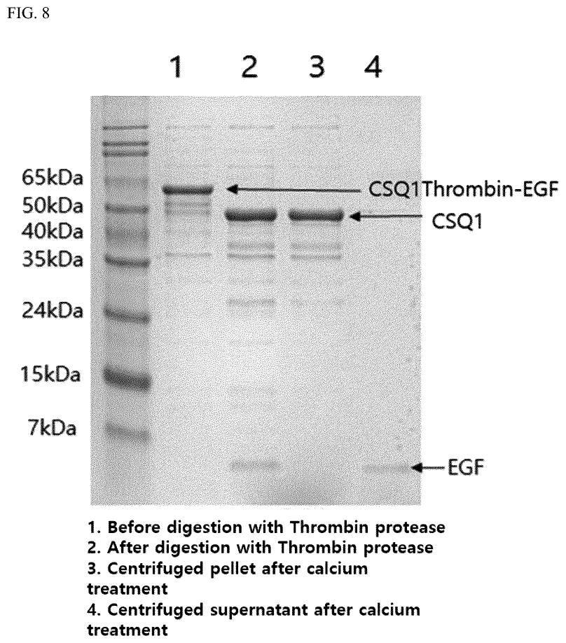

In order to isolate only EGF from CSQ1Thrombin-EGF protein, the recombinant protein was incubated overnight at 30° C., with 5 μl of thrombin protease. The resulting digests were run by SDS-PAGE (FIG. 8). As a result, it was observed that the CSQ-tag and the EGF fusion protein can be easily segregated using thrombin protease.

### Example 3: Separation of KGF1. VEGF, and FGF2 Using CSQ-Tag

3-1. Purification of CSQ1TEV-KGF1, CSQ1TEV-VEGF, and CSQ1TEV-FGF2 Genes of KGF1 (SEQ ID NO: 13), VEGF (SEQ ID NO: 14), and FGF2 (SEQ ID NO: 15) were synthesized in Bioneer Corporation and each cloned into CSQ1TEV which was then transformed into BL21 cells before being spread on agar plates containing ampicillin. Colonies grown on the agar plates were each inoculated into 5 ml of LB broth containing ampicillin (50 μg/ml) and cultured overnight at 37° C., while stirring at 200 rpm. The cell culture was mixed at a ratio of 1:1 with glycerol to give a 1-ml stock and stored at −80° C., in a deep-freezer. The stock was inoculated in an amount of 100 μl into 30 ml of LB broth containing ampicillin (50 μg/ml) and cultured overnight at 37° C., while stirring at 200 rpm. The culture was transferred to 1 L of LB broth containing ampicillin (50 μg/ml) and incubated until OD=0.6. Then, additional incubation was carried out overnight at 17° C., in the presence of 1 mM isopropyl-β-D-thiogalatopyranoside (IPTG) while stirring at 200 rpm. After centrifugation at 4° C., and 4000 rpm for 20 min, the supernatant was discarded and the cell pellet was suspended in a lysis buffer (20 mM Tris (pH 8.0), 300 mM NaCl, and 10 mM imidazole).

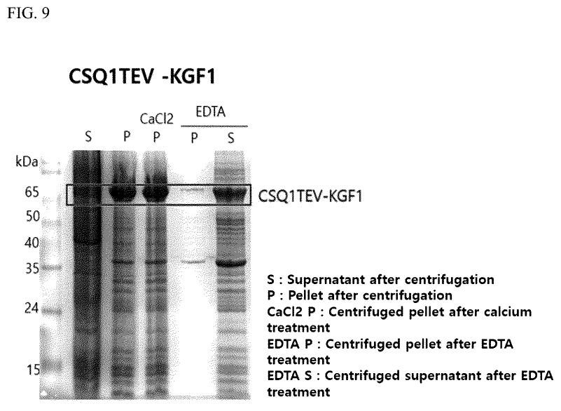

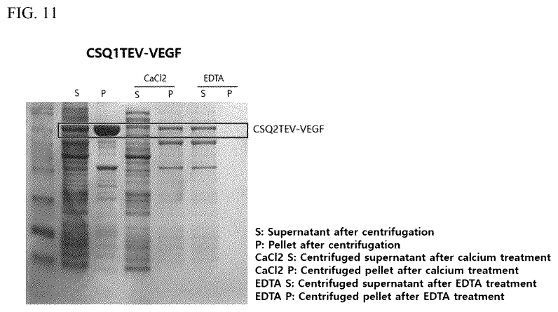

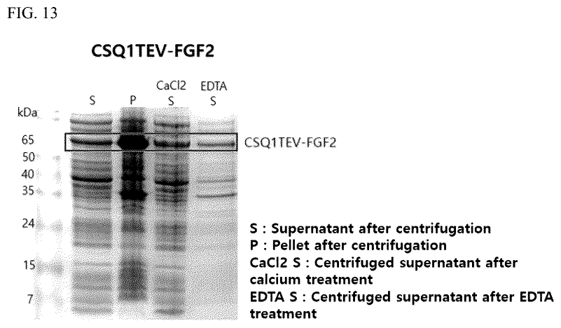

The suspension was stored overnight at −80° C., and then completely thawed, followed by adding a solution of 10 mg of PMSF (phenyl methane sulfonyl fluoride) in 1 ml of DMSO to 1 ml of the thawed E. coli. The E. coli was lysed using a sonicator, followed by centrifugation at 4° C., and 13,000 rpm for 1 hour. The supernatant was incubated with 20 mM CaCl₂) at 4° C., for 1 hour and then centrifuged at 5000 rpm for 30 min. After removal of the supernatant, the pellet thus formed was suspended in EDTA to obtain CSQ1TEV-KGF1, CSQ1TEV-VEGF, and CSQ1TEV-FGF2 proteins (FIGS. 9, 11, and 13).

### 3-2. KGF1, VEGF, and FGF2 Isolation Using TEV Protease

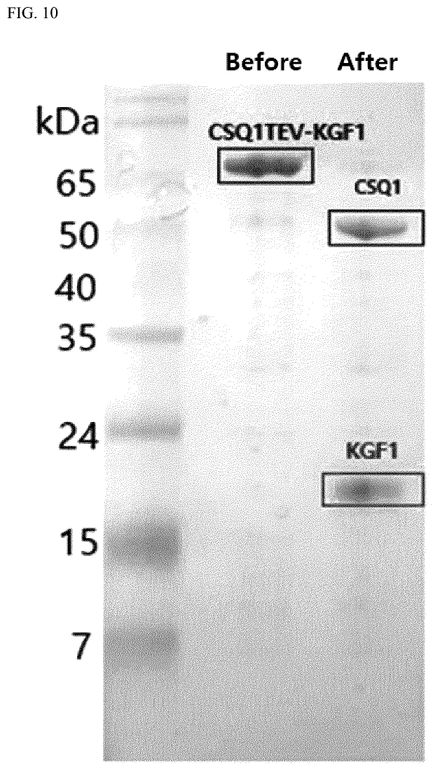

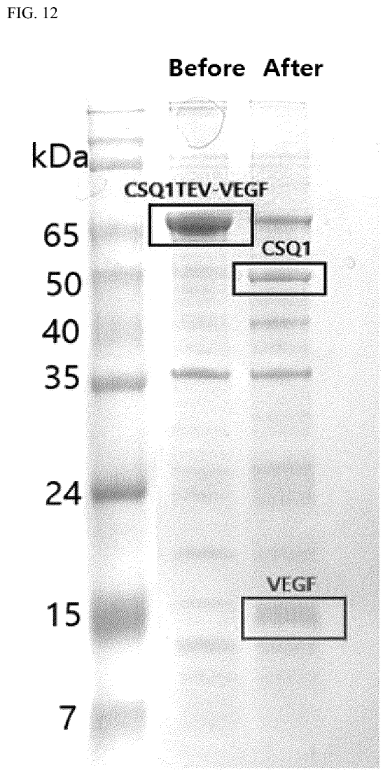

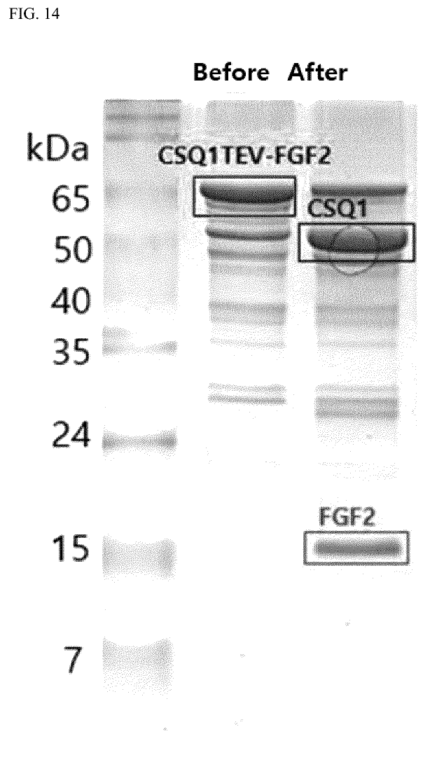

In order to isolate only KGF1 from CSQ1TEV-KGF1 protein (also from CSQ1TEV-VEGF and CSQ1TEV-FGF proteins), the recombinant protein was incubated overnight at 30° C. with 5 μl of TEV protease (Genscript, USA). The resulting digests were run by SDS-PAGE (FIGS. 10, 12, and 14). As a result, it was observed that segregation was easily made between CSQ-tag and KGF1 fusion protein, between CSQ-tag and VEGF protein, and between CSQ-tag and FGF protein, using TEV protease.

### Example 4: Separation of BMP2, TGFβ, HRP, and GLP1 Using CSQ-Tag

4-1. Cloning of BMP2 and TGFβ into TEVCSQ1 or ThrombinCSQ1 Vector

In order to clone BMP2 and TGFβ into TEVCSQ1 or ThrombinCSQ1 vector, the following oligonucleotides Amplify-F1 and Amplify-B1 were synthesized (Bioneer Corporation, Daejeon, Korea).

(SEQ ID NO: 24)
5′-CAGCAAGACAGCGATGGATCC-3′
(SEQ ID NO: 25)
5′-CGACTTACAGGTGATCTCGAG-3′

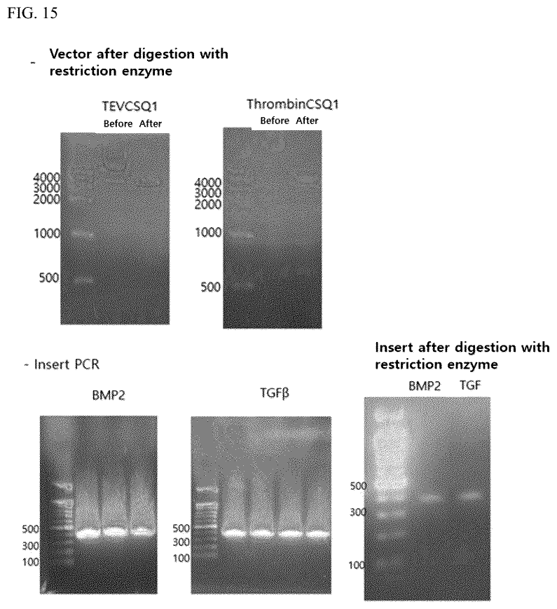

For use in amplification, Amplify-F1 20 pmol, Amplify-B1 20 pmol, PCR PreMix (Bioneer Corporation, Daejeon, Korea), and a template 10 μg were mixed and added with distilled water to form a final volume of 20 μl. The mixture was subjected to PCR (95° C., for 5 min. 30 cycles of: 95° C. 30 sec: 47° C., 30 sec; and 72° C. 45 sec, and 72° C. 5 min), followed by purification (PCR purification kit. GeneAll. Seoul. Korea) to obtain BMP2 (SEQ ID NO: 16) and TGFβ (SEQ ID NO: 17) genes. In order to insert the BMP2 gene (or TGFβ gene) into a TEVCSQ1 or ThrombinCSQ1 vector, the CSQ1TEV or CSQ1Thrombin vector and the insert DNA were treated with restriction enzymes. About 1 μg of the insert DNA was incubated overnight with BamHI (New England Biolabs (NEB). Ipswich) and then for two days with Xhol (NEB. Ipswich). Thereafter, the DNA digests were purified using a PCR purification kit. In addition, about 3 μg of the TEVCSQ or ThrombinCSQ vector was incubated for 3 hours with CIAP (Calf Intestinal Alkaline Phosphatase) (Takara. Japan). followed by purification with a PCR purification kit. The DNA insert was ligated to the CSQTEV or CSQ1Thrombin vector at room temperature for 3 hours in the presence of T4 DNA ligase (Bioneer Corporation. Daejeon. Korea) (FIG. 15).

Next, transformation was made of the DNA resulting from ligating the BMP2 insert to the TEVCSQ1 or ThrombinCSQ1 vector. After being thawed on ice. 100 μl of DH5a competent cells was mixed and reacted for 30 min with 5 μl of the ligate solution. Heat shock was performed at 42° C. for 30 sec on the reaction mixture which was then added with 200 μl of SOC medium and cultured at 37° C. for 40 min before being spread on plates. The colonies thus formed on the plate were randomly picked up. The cloned colonies were committed to sequencing in Bioneer Corporation. Genes of HRP (SEQ ID NO: 18) and GLP1 (SEQ ID NO: 19) were also synthesized in Bioneer Corporation and cloned into TEVCSQ1 in the same manner as described above.

### 4-2. Purification of BMP2-TEVCSQ1 or BMP2-ThrombinCSQ1, TGFβ-TEVCSQ1 or TGFβ-ThrombinCSQ1, HRP-TEVCSQ1, and GLP1-TEVCSQ1

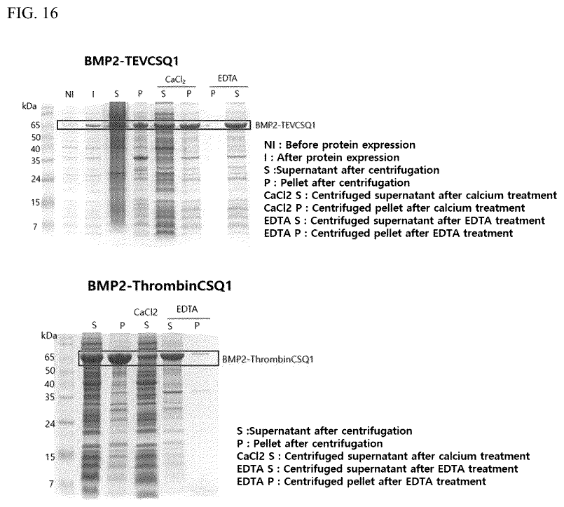

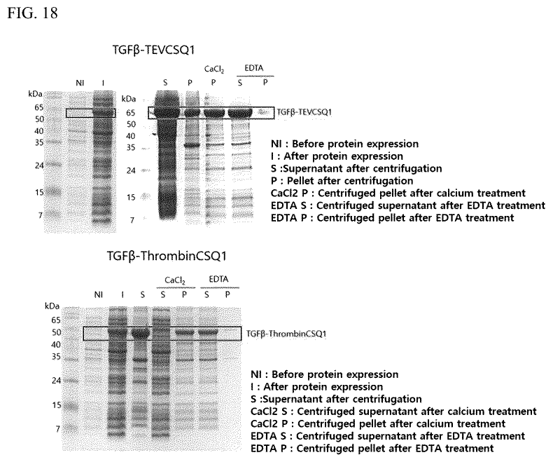

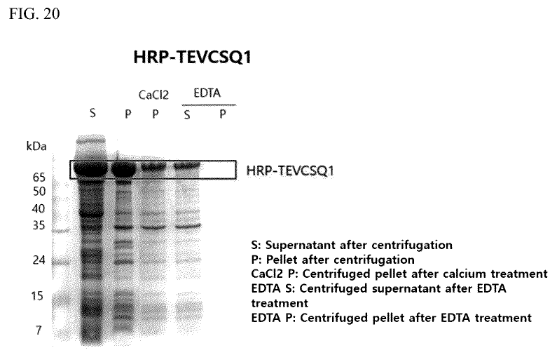

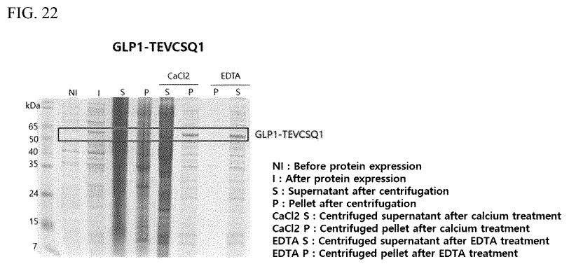

BMP2-TEVCSQ1. BMP2-ThrombinCSQ1. TGFβ-TEVCSQ1. TGFβ-ThrombinCSQ1. HRP-TEVCSQ1. GLP-TEVCSQ1 vectors sequenced in Bioneer Corporation were each transformed into BL21 cells which were then spread on agar plates containing ampicillin. Colonies grown on the agar plates were each inoculated into 5 ml of LB broth containing ampicillin (50 μg/ml) and cultured overnight at 37° C., while stirring at 200 rpm. The cell culture was mixed at a ratio of 1:1 with glycerol to give a 1-ml stock and stored at −80° C., in a deep-freezer. The stock was inoculated in an amount of 100 μl into 20 ml of LB broth containing ampicillin (50 μg/ml) and cultured overnight at 37° C., while stirring at 200 rpm. The culture was transferred to 1 L of LB broth containing ampicillin (50 μg/ml) and incubated until OD=0.6. Then, additional incubation was carried out overnight at 17° C., in the presence of 1 mM isopropyl-β-D-thiogalatopyranoside (IPTG) while stirring at 200 rpm. 35 After centrifugation at 4° C., and 4000 rpm for 20 min, the supernatant was discarded and the cell pellet was suspended in a lysis buffer (20 mM Tris (pH 8.0), 300 mM NaCl, and 10 mM imidazole). The suspension was stored overnight at −80° C., and then completely thawed, followed by adding a solution of 10 mg of PMSF (phenyl methane sulfonyl fluoride) in 1 ml of DMSO to 1 ml of the thawed E. coli. The E. coli was lysed using a sonicator before centrifugation at 4° C., and 13,000 rpm for 1 hour. The supernatant was incubated with 20 mM CaCl₂) at 4° C. for 1 hour and then centrifuged at 5000 rpm for 30 min. After removal of the supernatant, the pellet thus formed was suspended in EDTA to obtain BMP2-TEVCSQ1 and BMP2-ThrombinCSQ1 proteins (FIGS. 16, 18, 20, and 22).

### 4-3. Isolation of BMP2, TGFβ, HRP, and GLP1 Using TEV Protease

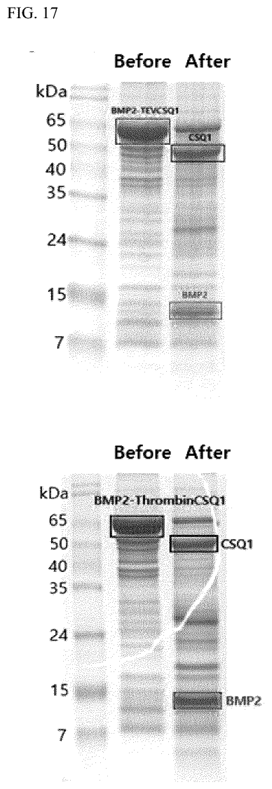

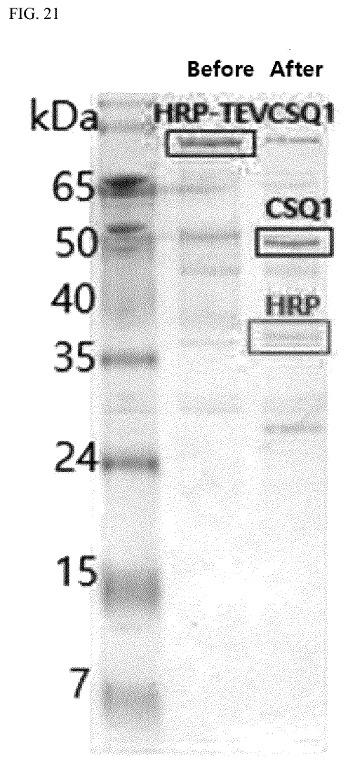

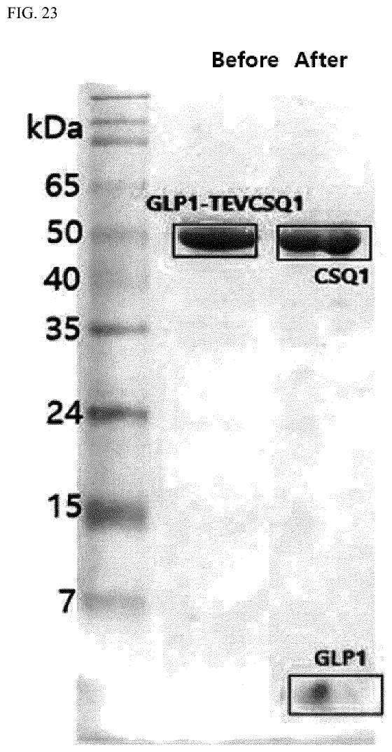

In order to isolate BMP2 from BMP2-TEVCSQ1 protein, the recombinant protein was incubated overnight at 30° C., with 2 μl of TEV protease (Genscript, USA) (the same experiments were carried out for TGFβ-TEVCSQ1, HRP-TEVCSQ1, and GLP1-TEVCSQ1 proteins). The resulting digests were run by SDS-PAGE (FIGS. 17, 19, 21, and 23). As a result, it was observed that the CSQ-tag and the BMP2 fusion protein can be easily segregated using TEV protease.

### 4-4. Isolation of BMP2 and TGFβ Using Thrombin Protease

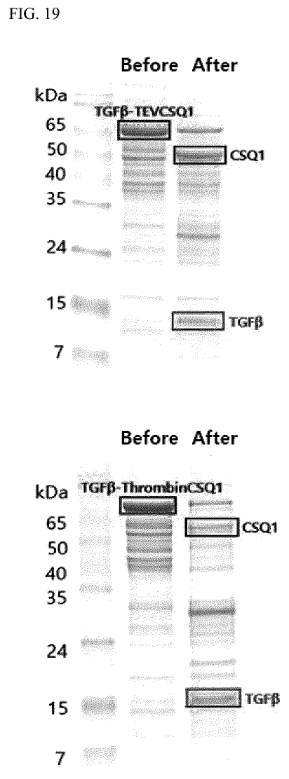

In order to isolate BMP2 from BMP2-ThrombinCSQ1 protein, the recombinant protein was incubated overnight at 22° C., with 5 μl of thrombin protease (the same procedure was also carried out for TGFβ-TEVCSQ1). The resulting digests were run by SDS-PAGE (FIGS. 17 and 19). As a result, it was observed that the CSQ-tag and the BMP2 fusion protein can be easily segregated using thrombin protease.

### Example 5: Separation of BMP2 and KGF1 Using CSQ-Tag

5-1. Cloning of BMP2 and KGF1 into CSQ2TEV or CSQ2Thrombin Vector

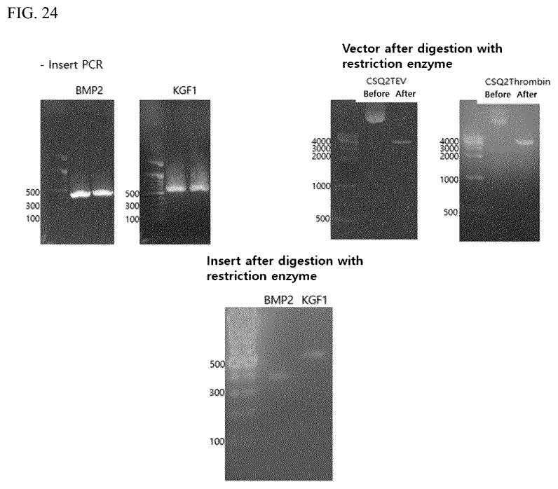

In order to clone BMP2 and KGF1 into CSQ2TEV or CSQ2Thrombin vector, Amplify-F1 and Amplify-B1 synthesized above (Bioneer Corporation, Daejeon, Korea) were employed. For use in amplification, Amplify-F1 20 pmol, Amplify-B1 20 pmol, PCR PreMix (Bioneer Corporation, Daejeon, Korea), and a template 10 μg were mixed and added with distilled water to form a final volume of 20 μl. The mixture was subjected to PCR (95° C., for 5 min, 30 cycles of: 95° C., 30 sec: 47° C., 30 sec; and 72° C., 45 sec, and 72° C., 5 min), followed by purification (PCR purification kit, GeneAll, Seoul, Korea) to obtain BMP2 (SEQ ID NO: 16) and KGF1(SEQ ID NO: 13) genes. In order to insert the BMP2 gene (or KGF1 gene) into a CSQ2TEV or CSQ2Thrombin vector, the CSQ2TEV or CSQ2Thrombin vector and the insert DNA were treated with restriction enzymes. About 1 μg of the insert DNA was incubated overnight with BamHI (New England Biolabs (NEB). Ipswich) and then for two days with Xhol (NEB. Ipswich). Thereafter, the DNA digests were purified using a PCR purification kit. In addition, about 3 μg of the CSQ2TEV or CSQ2Thrombin vector was incubated for 3 hours with CIAP (Calf Intestinal Alkaline Phosphatase) (Takara. Japan). followed by purification with a PCR purification kit. The DNA insert was ligated to the CSQ2TEV or CSQ2Thrombin vector at room temperature for 3 hours in the presence of T4 DNA ligase (Bioneer Corporation. Daejeon. Korea) (FIG. 24).

Next, transformation was made of the DNA resulting from ligating the BMP2 insert to the CSQ2TEV or CSQ2Thrombin vector. After being thawed on ice. 100 μl of DH5a competent cells was mixed and reacted for 30 min with 5 μl of the ligate solution. Heat shock was performed at 42° C., for 30 sec on the reaction mixture which was then added with 200 μl of SOC medium and cultured at 37° C. for 40 min before being spread on plates. The colonies thus formed on the plate were randomly picked up. The cloned colonies were committed to sequencing in Bioneer Corporation.

### 5-2. Purification of CSQ2TEV-BMP2 or CSQ2Thrombin-BMP2, and CSQ2TEV-KGF1 or CSQ2Thrombin-KGF1

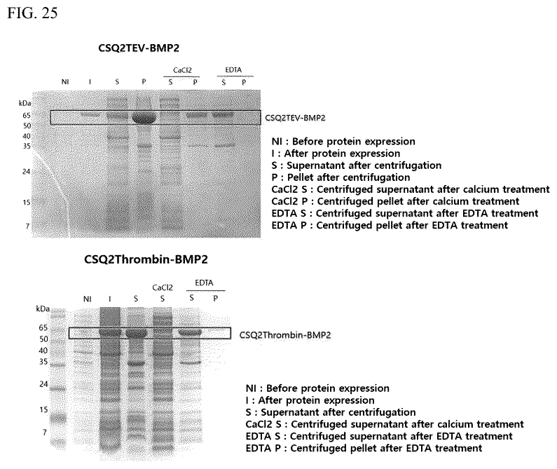

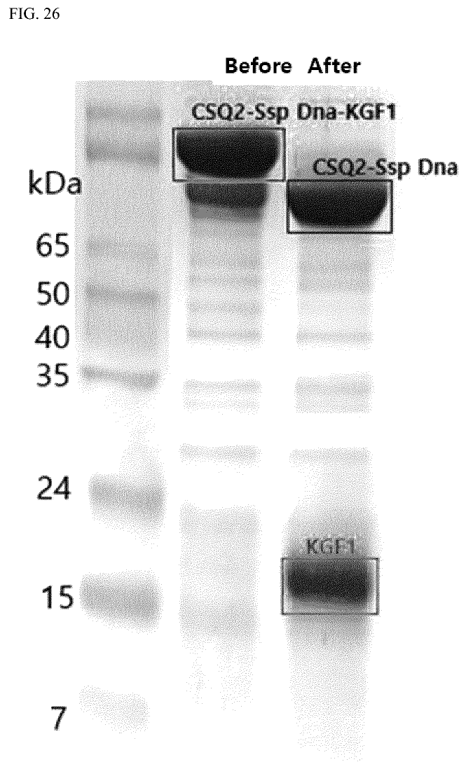

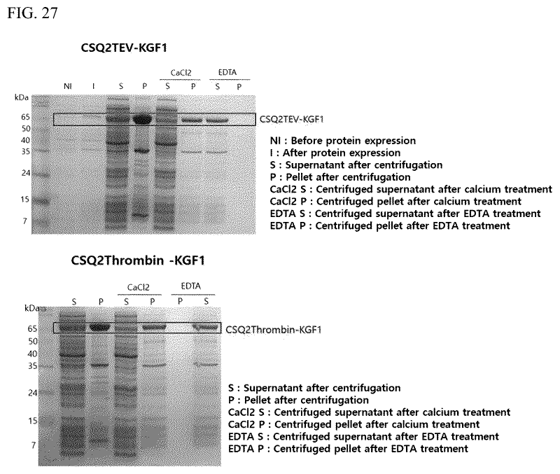

The DNAs sequenced in Bioneer Corporation were each transformed into BL21 cells which were then spread on agar plates containing ampicillin. Colonies grown on the agar plates were each inoculated into 5 ml of LB broth containing ampicillin (50 μg/ml) and cultured overnight at 37° C., while stirring at 200 rpm. The cell culture was mixed at a ratio of 1:1 with glycerol to give a 1-ml stock and stored at −80° C., in a deep-freezer. The stock was inoculated in an amount of 100 μl into 30 ml of LB broth containing ampicillin (50 μg/ml) and cultured overnight at 37° C., while stirring at 200 rpm. The culture was transferred to 1 L of LB broth containing ampicillin (50 μg/ml) and incubated until OD=0.6. Then, additional incubation was carried out overnight at 17° C., in the presence of 1 mM isopropyl-β-D-thiogalatopyranoside (IPTG) while stirring at 200 rpm. After centrifugation at 4° C., and 4000 rpm for 20 min, the supernatant was discarded and the cell pellet was suspended in a lysis buffer (20 mM Tris (pH 8.0). 300 mM NaCl, and 10 mM imidazole). The suspension was stored overnight at −80° C., and then completely thawed, followed by adding a solution of 10 mg of PMSF (phenyl methane sulfonyl fluoride) in 1 ml of DMSO to 1 ml of the thawed E. coli. The E. coli was lysed using a sonicator before centrifugation at 4° C., and 13.000 rpm for 1 hour. The supernatant was incubated with 20 mM CaCl₂) at 4° C., for 1 hour and then centrifuged at 5000 rpm for 30 min. After removal of the supernatant, the pellet thus formed was suspended in EDTA to obtain CSQ2TEV-BMP2 and CSQ2Thrombin-BMP2 proteins (FIGS. 25 and 27).

### 5-3. Isolation of BMP2 and KGF Using TEV Protease

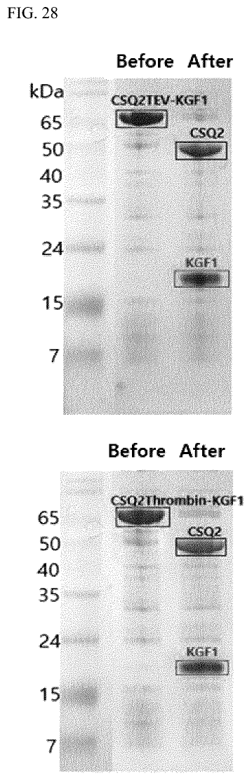

In order to isolate BMP2 from CSQ2TEV-BMP2 protein, the recombinant protein was incubated overnight at 30° C., with 2 μl of TEV protease (Genscript, USA) (the same experiments were carried out for CSQ2TEV-KGF1 protein). The resulting digests were run by SDS-PAGE (FIGS. 26 and 28). As a result, it was observed that the CSQ-tag and the BMP2 fusion protein can be easily segregated using TEV protease.

### 5-4. Isolation of BMP2 and KGF Using Thrombin Protease

In order to isolate BMP2 from CSQ2Thrombin-BMP2 protein, the recombinant protein was incubated overnight at 22° C., with 5 μl of thrombin protease (the same procedure was also carried out for CSQ2Thrombin-KGF1 protein). The resulting digests were run by SDS-PAGE (FIGS. 26 and 28). As a result, it was observed that the CSQ-tag and the BMP2 fusion protein can be easily segregated using thrombin protease.

### Example 6: Separation of HRP and GLP1 Using CSQ-Tag

### 6-1. Purification of HRP-TEVCSQ2 or HRP-ThrombinCSQ2 and GLP1-TEVCSQ2

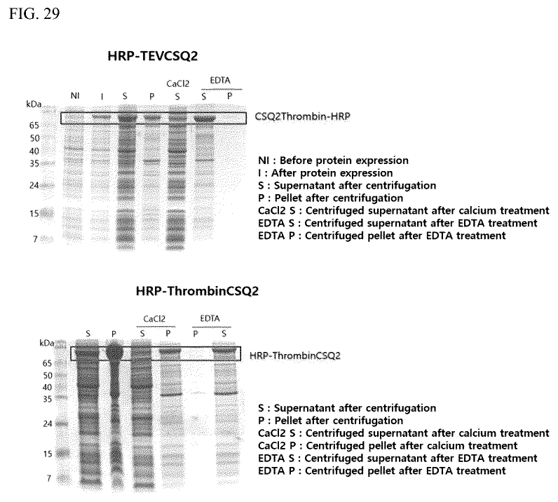

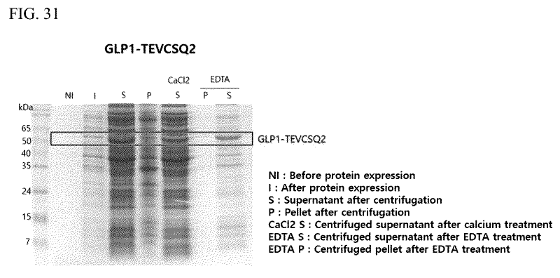

Genes of HRP (SEQ ID NO: 18) and GLP1 (SEQ ID NO: 19) sequenced in Bioneer Corporation were cloned into TEVCSQ2 and ThrombinCSQ2 vectors which were then transformed into BL21 cells, followed by spreading the cells on agar plates containing ampicillin. Colonies grown on the agar plates were each inoculated into 5 ml of LB broth containing ampicillin (50 μg/ml) and cultured overnight at 37° C., while stirring at 200 rpm. The cell culture was mixed at a ratio of 1:1 with glycerol to give a 1-ml stock and stored at −80° C., in a deep-freezer. The stock was inoculated in an amount of 100 μl into 30 ml of LB broth containing ampicillin (50 μg/ml) and cultured overnight at 37° C., while stirring at 200 rpm. The culture was transferred to 1 L of LB broth containing ampicillin (50 μg/ml) and incubated until OD=0.6. Then, additional incubation was carried out overnight at 17° C., in the presence of 1 mM isopropyl-β-D-thiogalatopyranoside (IPTG) while stirring at 200 rpm. After centrifugation at 4° C., and 4000 rpm for 20 min, the supernatant was discarded and the cell pellet was suspended in a lysis buffer (20 mM Tris (pH 8.0), 300 mM NaCl, and 10 mM imidazole). The suspension was stored overnight at −80° C., and then completely thawed, followed by adding a solution of 10 mg of PMSF (phenyl methane sulfonyl fluoride) in 1 ml of DMSO to 1 ml of the thawed E. coli. The E. coli was lysed using a sonicator before centrifugation at 4° C., and 13,000 rpm for 1 hour. The supernatant was incubated with 20 mM CaCl₂) at 4° C. for 1 hour and then centrifuged at 5000 rpm for 30 min. After removal of the supernatant, the pellet thus formed was suspended in EDTA to obtain HRP-TEVCSQ2, HRP-ThrombinCSQ2, and GLP1-TEVCSQ2 proteins (FIGS. 29 and 31).

### 6-2. Isolation of HRP and GLP1 Using TEV Protease

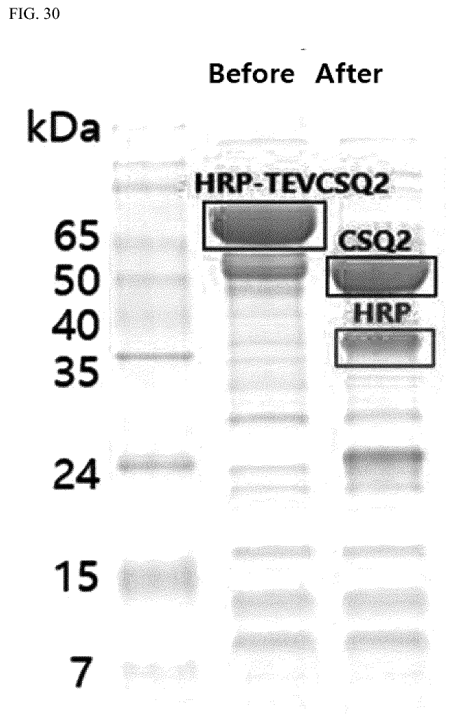

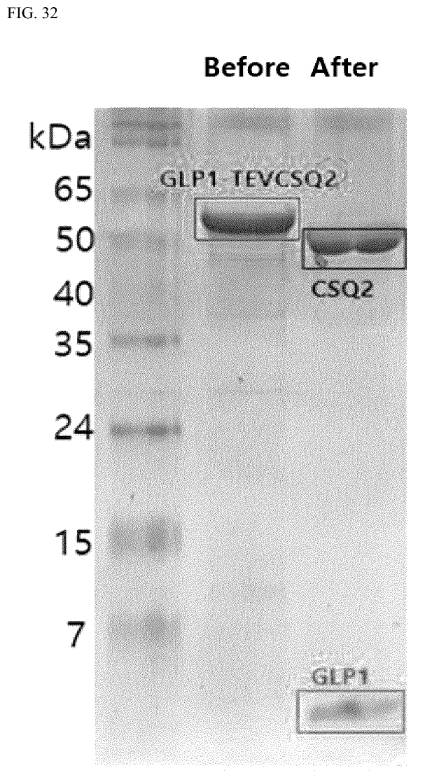

In order to isolate HRP from HRP-TEVCSQ2 protein, the recombinant protein was incubated overnight at 30° C., with 2 μl of TEV protease (Genscript, USA) (the same experiments were carried out for GLP1-TEVCSQ2 protein). The resulting digests were run by SDS-PAGE (FIGS. 30 and 32). As a result, it was observed that the CSQ-tag and the GLP1 fusion protein can be easily segregated using TEV protease.

### Example 7: Separation of EGF and KGF1 Using CSQ-Tag

### 7-1. Purification of CSQ1-Ssp Dna-EGF and CSQ2-Ssp Dna-KGF1

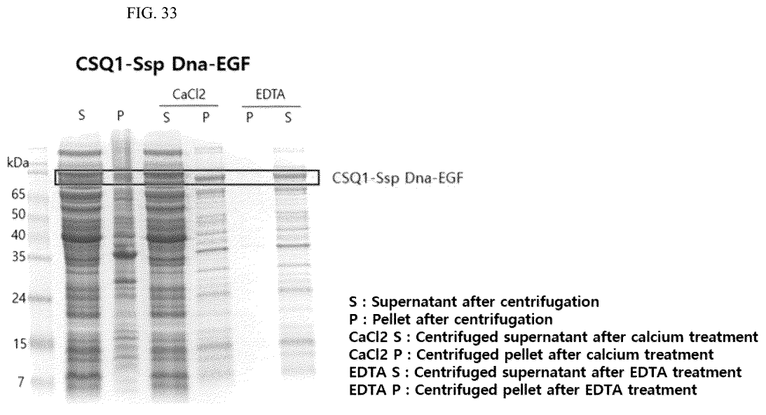

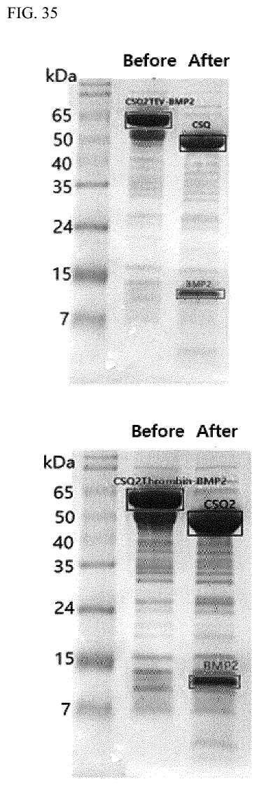

Genes of EGF (SEQ ID NO: 12) and KGF1(SEQ ID NO: 13) synthesized in Bioneer Corporation were cloned into CSQ1-Ssp Dna and CSQ2-Ssp Dna vectors which were then transformed into BL21 cells, followed by spreading the cells on agar plates containing ampicillin. Colonies grown on the agar plates were each inoculated into 5 ml of LB broth containing ampicillin (50 μg/ml) and cultured overnight at 37° C., while stirring at 200 rpm. The cell culture was mixed at a ratio of 1:1 with glycerol to give a 1-ml stock and stored at −80° C., in a deep-freezer. The stock was inoculated in an amount of 100 μl into 30 ml of LB broth containing ampicillin (50 μg/ml) and cultured overnight at 37° C., while stirring at 200 rpm. The culture was transferred to 1 L of LB broth containing ampicillin (50 μg/ml) and incubated until OD=0.6. Then, additional incubation was carried out overnight at 17° C., in the presence of 1 mM isopropyl-β-D-thiogalatopyranoside (IPTG) while stirring at 200 rpm. After centrifugation at 4° C., and 4000 rpm for 20 min, the supernatant was discarded and the cell pellet was suspended in a lysis buffer (20 mM Tris (pH 8.0), 300 mM NaCl, and 10 mM imidazole). The suspension was stored overnight at −80° C., and then completely thawed, followed by adding a solution of 10 mg of PMSF (phenyl methane sulfonyl fluoride) in 1 ml of DMSO to 1 ml of the thawed E. coli. The E. coli was lysed using a sonicator before centrifugation at 4° C., and 13,000 rpm for 1 hour. The supernatant was incubated with 20 mM CaCl₂) at 4° C. for 1 hour and then centrifuged at 5000 rpm for 30 min. After removal of the supernatant, the pellet thus formed was suspended in EDTA to obtain CSQ1-Ssp Dna-EGF and CSQ2-Ssp Dna-KGF1 proteins (FIGS. 33 and 35).

### 7-2. Isolation of EGF and KGF1 by pH

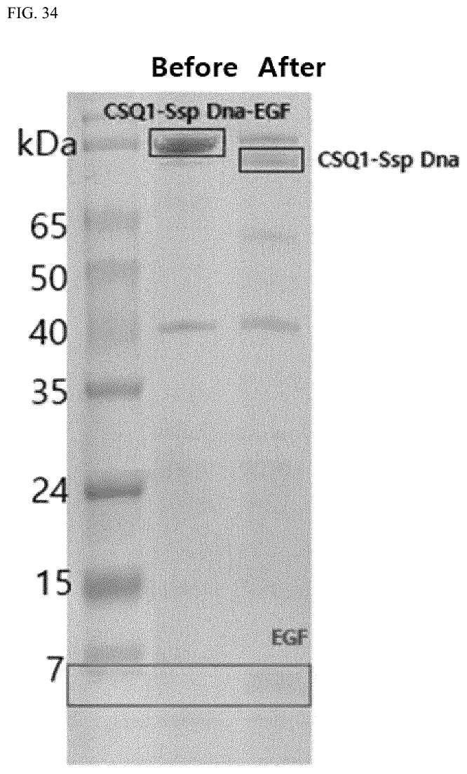

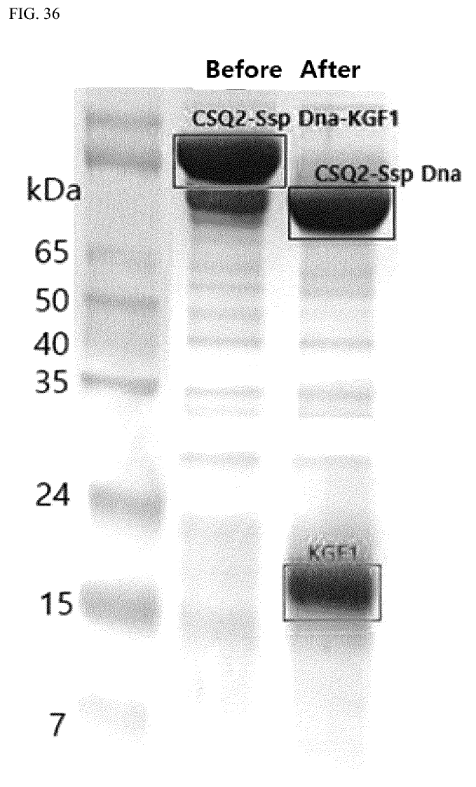

In order to isolate EGF from CSQ1-Ssp Dna-EGF protein, the recombinant protein was incubated overnight at room temperature with 20 mM HEPES pH 6.5, 500 mM NaCl buffer (the same experiments were carried out for CSQ2-Ssp Dna-KGF1 protein). The resulting reaction mixture was run by SDS-PAGE (FIGS. 34 and 36). As a result, it was observed that the CSQ-tag and the EGF fusion protein can be easily segregated by pH.

### Example 8: Separation of EGF and BMP2 Using CSQ-Tag

### 8-1. Purification of EGF-GyrA-CSQ1 and BMP2-GyrA-CSQ2

Genes of EGF (SEQ ID NO: 12) and BMP2 (SEQ ID NO: 16) synthesized in Bioneer Corporation were cloned into GyrA-CSQ1 and GyrA-CSQ2 vectors which were then transformed into BL21 cells, followed by spreading the cells on agar plates containing ampicillin.

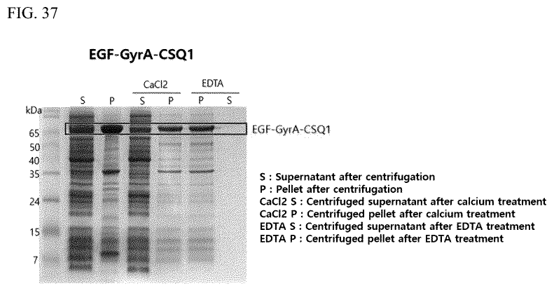

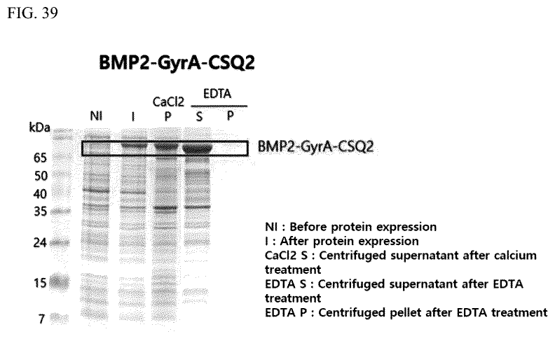

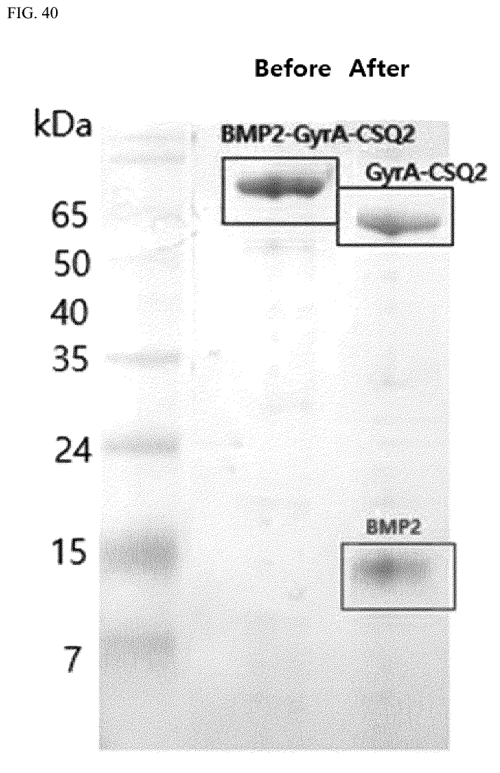

Colonies grown on the agar plates were each inoculated into 5 ml of LB broth containing ampicillin (50 μg/ml) and cultured overnight at 37° C., while stirring at 200 rpm. The cell culture was mixed at a ratio of 1:1 with glycerol to give a 1-ml stock and stored at −80° C., in a deep-freezer. The stock was inoculated in an amount of 100 μl into 30 ml of LB broth containing ampicillin (50 μg/ml) and cultured overnight at 37° C., while stirring at 200 rpm. The culture was transferred to 1 L of LB broth containing ampicillin (50 μg/ml) and incubated until OD=0.6. Then, additional incubation was carried out overnight at 17° C., in the presence of 1 mM isopropyl-β-D-thiogalatopyranoside (IPTG) while stirring at 200 rpm. After centrifugation at 4° C., and 4000 rpm for 20 min, the supernatant was discarded and the cell pellet was suspended in a lysis buffer (20 mM Tris (pH 8.0), 300 mM NaCl, and 10 mM imidazole). The suspension was stored overnight at −80° C., and then completely thawed, followed by adding a solution of 10 mg of PMSF (phenyl methane sulfonyl fluoride) in 1 ml of DMSO to 1 ml of the thawed E. coli. The E. coli was lysed using a sonicator before centrifugation at 4° C., and 13,000 rpm for 1 hour. The supernatant was incubated with 20 mM CaCl₂) at 4° C. for 1 hour and then centrifuged at 5000 rpm for 30 min. After removal of the supernatant, the pellet thus formed was suspended in EDTA to obtain EGF-GyrA-CSQ1 and BMP2-GyrA-CSQ2 proteins (FIGS. 37 and 39).

### 8-2. Segregation of EGF and BMP2 Using Dithiothreitol (DTT)

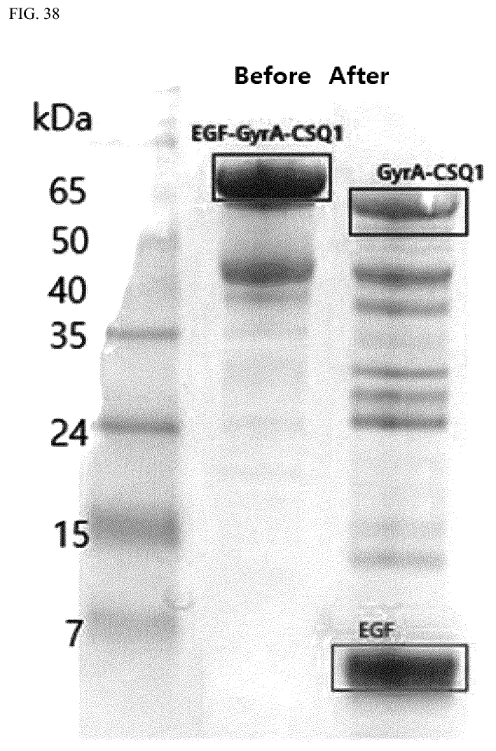

In order to isolate EGF from EGF-GyrA-CSQ1 protein, the recombinant protein was incubated overnight at room temperature with 20 mM HEPES pH 8.5, 500 mM NaCl, 40 mM DTT buffer (the same experiments were carried out for BMP2-GyrA-CSQ2 protein). The resulting reaction mixture was run by SDS-PAGE (FIGS. 38 and 36). As a result, it was observed that the CSQ-tag and the EGF fusion protein can be easily segregated using DTT.

Although the preferred embodiments of the present disclosure have been disclosed for illustrative purposes, those skilled in the art will appreciate that various modifications, additions and substitutions are possible, without departing from the scope and spirit of the present disclosure as disclosed in the accompanying claims.

### Example 9: Purification of CSQ-BMP2 Using Various Metals

The purification of CSQ1TEV-BMP2 using various metal ions (Ca2+, Mg2+, Zn2+, Ni2+, Mn2+, Cu2+, Ba2+, Sr2+, La3+, K+) was investigated. After synthesizing the BMP2 gene in Bioneer Co, it was cloned into the CSQ1TEV vector and transformed into BL21 cells, then spread onto LB agar plates containing ampicillin. Colonies grown on LB plates were inoculated into 5 mL of LB medium containing ampicillin (50 μg/mL) and cultured overnight at 37° C., and 200 rpm. The culture was mixed with glycerol at a 1:1 ratio to make a 1 mL stock solution, which was stored at −80° C.

A 100 μL aliquot of the stock was inoculated into a 30 mL LB medium containing ampicillin (50 μg/mL) and cultured overnight at 37° C., and 200 rpm. The resulting culture was then transferred to a IL LB medium containing ampicillin (50 μg/mL) and cultured until OD reached 0.6. Then, 1 mM isopropyl-B-D-thiogalactopyranoside (IPTG) was added, and the culture was incubated at 17° C. and 200 rpm for 24 hours.

After centrifuging the culture at 4000 rpm for 20 minutes at 4° C., the pellet was collected after discarding the supernatant. The pellet was resuspended in lysis buffer (20 mM Tris pH 8.0, 300 mM NaCl, and 10 mM imidazole), followed by freezing at −80° C., for one day. After thawing, 10 mg of PMSF (Phenyl methane sulfonyl fluoride) dissolved in 1 mL of DMSO was added to the E. coli lysate. After sonicating the E. coli and centrifuging at 13,000 rpm for one hour at 4° C., the supernatant was collected.

To the supernatant, various metal ions (Ca2+, Mg2+, Zn2+, Ni2+, Mn2+, Cu2+, Ba2+, Sr2+, La3+, K+) were added to a final concentration of 3 mM, and the mixture was allowed to react at 4° C. for 30 minutes. The solution was then centrifuged at 5000 rpm for 30 minutes to obtain purified CSQ1TEV-BMP2, with impurities removed.

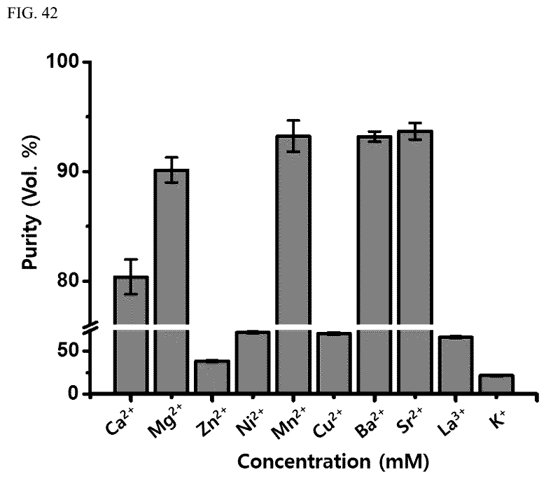

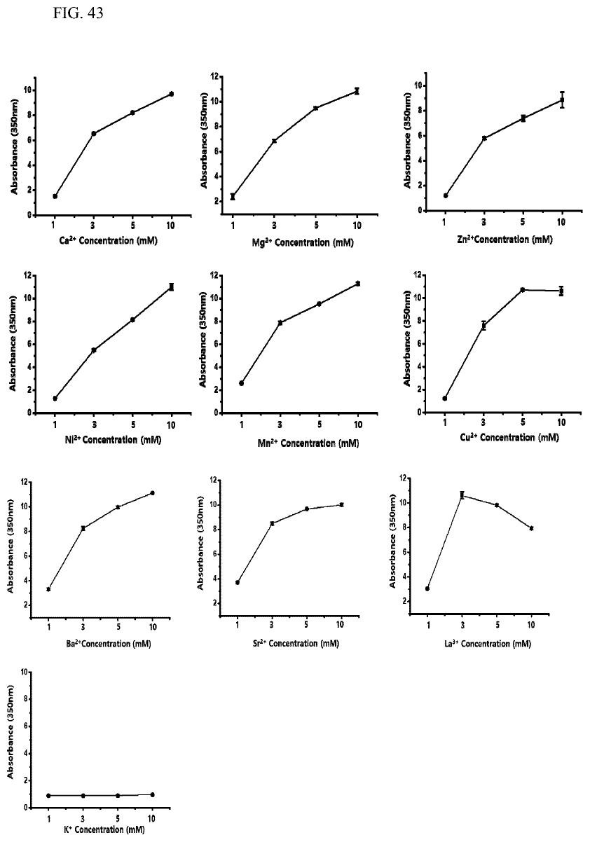

FIGS. 41 and 42 shows the results of CSQ1TEV-BMP2 protein purification with various metal ions, along with purity graphs. The purity graphs below were calculated by analyzing SDS-PAGE using Fusion FX analysis equipment and Evolution-capt Edge software (Vilber, Collegien, France)

### Example 10. Analysis of Turbidity Change of CSQ-BMP2 after Treatment of Various Metal Ions

To evaluate the formation of particle by CSQ1TEV-BMP2 protein in response to Ca2+, Mg2+, Zn2+, Ni2+, Mn2+, Cu2+, Ba2+, Sr2+, La3+, and K+ ions, various concentrations of metal ions (1 mM, 3 mM, 5 mM, 10 mM) were added to CSQ1TEV-BMP2. After storing at 4° C. for 30 minutes, turbidity was measured at 350 nm UV-vis spectrophotometer. The results confirmed that CSQ1TEV-BMP2 protein forms particle stably as turbidity increases with various metal ion concentrations (Ca2+, Mg2+, Zn2+, Ni2+, Mn2+, Cu2+, Ba2+, Sr2+, La3+, excluding K+)-(FIG. 43)

FIG. 43 shows the results of turbidity measurements of CSQ1TEV-BMP2 after reacting with various metal ions (Ca2+, Mg2+, Zn2+, Ni2+, Mn2+, Cu2+, Ba2+, Sr2+, La3+, K+).

Precipitation-based purification of CSQ-BMP2 was possible with 2+ and 3+ metal ions except K+, especially confirming excellent purification purity with Ca2+(purity=80.3%), Mg2+(purity=90.1%), Mn2+(purity=93.2%), Ba2+(purity-93.6%), and Sr2+(purity=93.1%). Moreover, Mg2+, Mn2+, Ba2+, and Sr2+ exhibited significantly better purification purity than the previously used Ca2+. Mn2+(purity=93.2%), Ca2+(purity=80.3%), Mg2+(purity=90.1%), Ba2+(purity=93.6%), and Sr2+(purity=93.1%) demonstrated excellent purification performance. Interestingly, Mn2+, Ba2+, Sr2+, and Mg2+ exhibited better purification purity than the previously used Ca2+.

1. A fusion protein, comprising:
a CSQ tag; and
a protein of interest,
wherein the CSQ tag acts to improve expression and water solubility of the protein of interest.

2. The fusion protein of claim 1, wherein the CSQ tag is coded for by an amino acid sequence of SEQ ID NO: 1 or 2.

3. The fusion protein of claim 1, wherein the CSQ tag is encoded by a nucleotide sequence of SEQ ID NO: 3 or 4.

4. The fusion protein of claim 1, wherein the CSQ tag and the protein of interest are fused to each other via a hydrolase-cleavable peptide composed of an amino acid sequence selected from the group consisting of SEQ ID NOS: 5 to 8.

5. The fusion protein of claim 1, wherein the protein of interest comprises at least one selected from the group consisting of a polymer protein, a glycoprotein, a cytokine, a growth factor, a blood factor, a vaccine, a hormone, an enzyme, and an antibody.

6. The fusion protein of claim 1, wherein the protein of interest comprises at least one selected from the group consisting of interleukin-2, blood clotting factor VII, blood clotting factor VIII, blood clotting factor IX, an immunoglobulin, horseradish peroxidase (HRP), a cytokine, α-interferon, β-interferon, γ-interferon, colony stimulating factor (GM-CSF), human fibronectin extra domain B (EBD), bone morphogenetic protein (BMP), fibroblast growth factor (FGF), vascular endothelial growth factor (VEGF), nerve growth factor (NGF), epidermal growth factor (EGF), insulin-like growth factor (IGF), trans-forming growth factor-α and -β (TGF-α and -β), brain-derived neurotrophic factor (BDNF), platelet-derived growth factor (PDGF), placental growth factor (P1GF), hepatocyte growth factor (HGF), fibroblast growth factor 1 and 2 (FGF-1 and -2), keratinocyte growth factor (KGF), glucagon-like peptide-1 (GLP-1), exendin, somatostatin, LHRH (luteinizing hormone-releasing hormone), adrenocorticotropic hormone, growth hormone-releasing hormone, oxytocin, thymosin alpha-1, corticotropin-releasing factor, calcitonin, bivalirudin, vasopressin, phospholipase-activating protein (PLAP), insulin, tumor necrosis factor (TNF), follicle-stimulating hormone, thyroid-stimulating hormone, antidiuretic hormone, pigmentary hormone, parathyroid hormone, luteinizing hormone, calcitonin gene-related peptide (CGRP), enkephalin, somatomedin, erythropoietin, hypothalamic releasing factor, prolactin, chorionic gonadotropin, tissue plasminogen activator, growth hormone releasing peptide (GHPR), thymic humoral factor (THF), asparaginase, arginase, arginine deiminase, adenosine deaminase, peroxidase dismutase, endotoxinase, catalase, chymotrypsin, lipase, uricase, adenosine diphosphatase, tyrosinase, bilirubin oxidase, glucose oxidase, glucosidase, galactosidase, glucocerebrosidase, and glucuronidase.

7. The fusion protein of claim 1, wherein the protein of interest is coded for by an amino acid sequence selected from the group consisting of SEQ ID NOS: 9 to 19.

8. A nucleic acid, including a nucleotide sequence coding for the fusion protein of claim 1.

9. An expression vector, carrying the nucleic acid of claim 8.

10. A cell, transformed with the expression vector of claim 9.

11. The cell of claim 10, wherein the cell is Escherichia coli, Bacillus subtilis, Bacillus thuringiensis, Salmonella typhimurium, Serratia marcescens, Pseudomonas spp. yeast, insect cells, CHO (Chinese hamster ovary) cells, W138, BHK, COS-7, 293, HepG2, 3T3, RIN, MDCK cells, or plant cells.

12. A method for expressing and purifying a protein of interest by using a CSQ tag, the method comprising:
A) constructing an expressing vector carrying a nucleic acid coding for the fusion protein of claim 1;
B) transducing the expression vector into a host cell to obtain a transformant;
C) expressing the fusion protein including the CSQ tag and the protein of interest in the transformant;
D) precipitating the fusion protein including the CSQ tag and the protein of interest from the transformant in presence of at least one metal ion selected from the group consisting of manganese, barium, strontium, lanthanum, nickel, magnesium, copper, zinc and calcium; and
E) separating the protein of interest from the fusion protein with a hydrolase.

13. The method according to claim 12, wherein said at least one metal is calcium.

14. The method according to claim 12, wherein said at least one metal comprises Mg2+.

15. The method according to claim 12, wherein said at least one metal comprises Mn2+.

16. The method according to claim 12, wherein said at least one metal comprises Ba2+.

17. The method according to claim 12, wherein said at least one metal comprises Sr2+.

18. The method according to claim 12, wherein said at least one metal comprises La3+.

19. The method according to claim 12, wherein said at least one metal comprises Cu2+.

20. The method according to claim 12, wherein said at least one metal comprises Ni2+.

21. The method according to claim 12, wherein said at least one metal comprises Zn2+.

22. The method according to claim 12, wherein said at least one metal excludes calcium.
